(19) United States
(12) Patent Application Publication
(10) Pub. No.: US 2004/0048926 A1
(43) Pub. Date: Mar. 11, 2004

(54) Use of docosahexaenoic acid and arachidonic acid to enhance the visual development of term infants breast-fed up to the age of six months

(76) Inventors: Dennis Robert Hoffman, Dallas, TX (US); Eileen Elizabeth Birch, Dallas, TX (US); Julia Anne Boettcher, Evansville, IN (US); Deborah Ann Schade, Evansville, IN (US)

Correspondence Address: Nelson Mullins Riley & Scarborough LLP, Keenan Building, Third Floor, 1330 Lady Street, Columbia, SC, 29201, US

(21) Appl. No.: 10/387,966

(22) Filed: Mar. 13, 2003

Related U.S. Application Data
(60) Provisional application No. 60/365,221, filed on Mar. 15, 2002.

(51) Int. Cl. A61K 31/202

(52) U.S. Cl. 514/560

(57) ABSTRACT

A method for enhancing the visual development of term infants who have been breast-fed for a number of months, up to six months of age or later, involving the administration to those infants of a combination of docosahexaenoic acid and arachidonic acid from the time of weaning.

### CROSS-REFERENCE TO RELATED APPLICATIONS

The present application claims the benefit of U.S. Provisional Application Serial No. 60/365,221 filed Mar. 15, 2002, which is incorporated herein by reference thereto.

### STATEMENT REGARDING FEDERALLY SPONSORED RESEARCH

The U.S. Government has a paid-up license in this invention and the right in limited circumstances to require the patent owner to license others on reasonable terms as provided for by terms of Grant No. HD22380 awarded by the National Institute of Health.

### BACKGROUND OF THE INVENTION

1. Field of the Invention

The present invention relates generally to methods to enhance the visual development of term infants and, more particularly, of term infants that have been breast-fed for a number of months, including infants breast-fed up to the age of six months or later, before weaning to formula. The present invention also relates to the supplementation with docosahexaenoic acid and arachidonic acid of infant formulas designed for term infants who have been breast-fed for a number of months, including infants breast-fed up to the age of six months or later.

2. Description of Related Art

The importance of adding certain polyunsaturated fatty acids (PUFA) such as alpha-linolenic acid (“LNA”) and linoleic acid (“LA”) to infant formulas is generally recognized, and the addition of LA mandatory under current federal regulations. The art, until recently, has not concluded that other specific long-chain polyunsaturates (LCP) such as docosahexaenoic acid (DHA) and arachidonic acid (ARA) should also be added to infant formulas, particularly to formulas designed for term infants. The present invention demonstrates that term infants that are breast-fed for a number of months, including infants breast-fed up to the age of six-months or later, and then weaned to a DHA- and ARA-supplemented formula show enhanced visual development.

Lipids or fats, the source of fatty acids, are a basic component of human nutrition. Initially, lipids were thought to be only a source of energy for growth and metabolism. However, they are now recognized for their role in cell structure development and function and, particularly, in the development of an infant's nervous tissue. See, Crozier, G. L. et al., Monarsschr Kinderheilkd 1995, 143:S95-98.

Fatty acids are carboxylic acids with a carbon chain having a carboxyl group, (—COOH), at one end of the molecule, the alpha end, and a methyl group, (—CH3), at the other end, the omega (ω) or “n” end. These acids are characterized by the saturation and length of the carbon chain. Carbon-to-carbon bonds may be single (saturated bonds) or double (unsaturated bonds). Fatty acids are polyunsaturated if they have more than one double bond in the chain. In addition, fatty acids are called short-chain, middle-chain, or long-chain acids depending on the number of carbons in the chain. Short-chain fatty acids have from 2 to about 6 carbons in the chain; medium chain acids have more than 6 to about 12 carbons in the chain; and long-chain fatty acids have from more than about 14 to 24 or more carbons in the chain. Thus, a fatty acid is represented by a sequence of three numbers: the first number indicates the number of carbons in the chain; the second number indicates the number of double bonds in the chain; and the third number indicates the position of the first carbon having a double bond counting from the ω or “n” end of the chain. Thus, for example, the alpha-linolenic fatty acid is represented as (18:3ω3) or (18:3n-3) which indicates that the acid has 18 carbons and 3 double bonds in the carbon chain, and that the first carbon having a double bond is in the third position counting from the ω or “n” end of the chain.

Of the PUFAs, both alpha-linolenic acid (18:3ω3; LNA) and linoleic acid (18:2ω6; LA) are now regarded as nutritionally essential acids. See, Lauritzen, L. et al., Progress in Lipid Research 2001; 40:1-94; see also Hansen, D. R., Trends Biochem Sci 1986; 11: 263-5; see also Holman, R T., J Nutr 1998; 128:S427-33; see also Neuringer, M. et al., Annual Rev Nutr 1988; 8:517-41; Birch, E. E, et al., Pediatric Research 1998; 44:201-209. These fatty acids are identified as nutritionally essential because, though they play a critical role in metabolism, the human body cannot synthesize them and, thus, they must be provided for as part of the human diet to support normal health and development. De novo or “new” synthesis of the ω-3 and ω-6 essential fatty acids does not occur in the human; however, the body can convert these fatty acids to LCPs such as DHA and ARA although at very low efficiency. For this reason, federal regulations mandate that linoleic acid (LA) be added to infant formulas. Both acids must be part of the nutritional intake since the human body cannot insert double bonds closer to the omega end than the seventh carbon atom counting from that end of the molecule. Thus, all metabolic conversions occur without altering the omega end of the molecule that contains the ω-3 and ω-6 double bonds. Consequently, ce3 and ω-6 acids are two separate families of essential fatty acids since they are not interconvertible in the human body. See Lauritzen et al. (2001), opus cit.

The last trimester of prenatal development and the early postnatal months are periods of rapid maturation of the photoreceptors and of rapid increase in the number of synapses and dendritic arborizations in the brain. See Birch et al. (1998), opus cit., and citations therein. These processes require the deposition of lipids, particularly ω-3 and ω-6 LCPs, in neuronal membranes. See Id. Limitation in the supply of LCPs may modify the growth and function of the central nervous system since the quantity and quality of the LCPs incorporated into neural membranes influence their physical and functional properties. See Id.

Two members of the ω-3 and ω-6 families of fatty acids, docosahexaenoic acid (22:6ω3; DHA) and arachidonic acid (20:4ω6; ARA), are of particular interest in infant nutrition because they are found in high concentrations in the brain (see, Sastry, P. S., Progress in Lipid Research 1985; 24:69-176) and the retina (see, Fliesler, S. L. et al., Progress in Lipid Research 1983; 22:79-131.) During the infant's first year of life, there is a five-fold increase in the total number of neural synapses in the human striate cortex. See, Huttenlocher, P. et al., Human Neurobiol 1987; 6:1-9. During the same period, there is also a five-fold accumulation of DHA in the human forebrain. See, Martinez, M., J Pediatr 1992; 120:S129-138. Typically, DHA is present in membranes throughout the body at levels of 1 to 4% of total fatty acids; however, higher levels of 9%, 25%, 20% and 35% are found in the neural cortex, neural synapses, the retina, and rod receptors outer segments, respectively. See Martinez, M. (1992), opus cit.; Cotman, C. et al., Biochemistry 1969; 8:4606-12; see also Fliesler et al. (1983), opus cit. Cunnane et al. calculated that in the first six months of life, the brain accumulates an average of 5.1 mg of DHA per day in breast-fed infants, about twice the accretion rate in formula-fed infants (2.5 mg DHA per day). See, Cunnane, S. C. et al., Lipids 2000; 35:105-11.

Clinical studies of infant formula composition have introduced evidence that the presence of DHA in an infant's nutritional intake may confer an advantage in the infant's cognitive development. The presence of DHA in the diet of pre-term or term infants has been associated with higher mental development scores (measured on the Mental Development Index (MDI) of the Bayley Scales of Infant Development) (see, Carlson, S. E., World Review of Nutrition and Diet 1994; 75:63-9; see also, Damli, A. et al., in: Carlson, S. et al. (editors), Infant Nutrition: Consensus and Controversies. Champaign, Ill. American Oil Chemists' Society, p.14 (1996); see also, Birch, E. E. et al., Developmental Medicine and Child Neurology 2000;42:174-181.), higher psychomotor development scores (Brunet-Lezine Test) (see, Agostoni, C. et al., Pediatric Research 1995; 38:262-6), shorter-look duration to novel stimuli on the Fagan Test (see, Carlson, S. E., Lipids 1996; 31:85-90), and better problem solving skills (Infant Planning Test) (see, Willatts, P., Lancet 1998; 352:688-91).

In addition to providing dietary ω-3 fatty acids, i.e., the DHA family of fatty acids, there is a need to maintain a balance with ω-6 fatty acids, i.e., the ARA family of fatty acids, since there is competition between ω-3 and ω-6 fatty acids for incorporation into circulating lipids and cellular membranes. See, Lands, W. E. M. et al., Lipids 1990; 25: 505-516. ARA is a bioactive fatty acid, the primary precursor of eicosanoids and prostaglandins and, as such, participates in immune and inflammatory responses. The importance of ARA is further substantiated by reports that blood lipid levels of ARA are correlated with growth in neonates. See, Carlson, S. E. et al., Proc Natl Acad Sci 1993; 90:1073-77; see also, Koletzko, B. et al., Lipids 1996; 31: 79-83.

The Carlson et al.'s and Koletzko et al.'s studies showed that preterm and term infants fed with DHA-supplemented formula show an improvement in those parameters associated with visual function and mental development. However, the same infants had reduced ARA levels in red blood cell (RBC) membranes and exhibited poorer growth when compared to preterm infants fed with unsupplemented formula. Since the fish oil used as the DHA source in the Carlson study also contained high levels of eicosapentaenoic acid (“EPA” 20:5ω3), an ARA competitor in many biochemical reactions, it was hypothesized that these high levels were responsible for the reduction in ARA levels and the poorer rate of physical growth. However, there is also preliminary evidence that DHA supplementation with low EPA fish oil may also adversely affect growth in preterm infants. See, Carlson, S. E. et al., Am J Clin Nutr 1996, 63:687-97; see also, Ryan, A. S. et al., Am J Human Biology 1999,11:457-67. Regardless of the cause of the growth depression, it has been shown that dietary ARA could not only restore the growth of preterm infants fed with DHA-supplemented formulas to the levels of preterm infants fed with formulas without DHA and ARA, but also enhances that growth beyond the levels achievable with formulas without DHA and ARA. See, Diersen-Schade, D. A. et al., PCT Intl Application, Intl Pub No.: WO 98/44917 (1998).

During the last trimester of fetal development, the fetus receives DHA and ARA from the mother by preferential transport across the placenta. See, Dutta-Roy, A. K., Am J Clin Nutr 2000; 71:315S-322S. Preterm infants are deprived of much of this DHA and ARA supply.

Postnatally, breast-fed infants receive a direct supply of DHA and ARA from the mother's milk. In standard infant formulas in the U.S., linoleic acid (LA) is the only required PUFA additive. DHA and ARA may be endogenously produced within the human body from the essential fatty acids, through alternating enzymatic desaturation and elongation, and they accumulate rapidly in the neural tissue during the last months of gestation and the first months of postnatal life. See, Makrides, M. et al., Am J Clin Nutr 1994; 60:189-94. However, there is considerable debate as to whether the infant can convert sufficient amounts of DHA and ARA from the essential fatty acids to meet the needs of a rapidly maturing infant.

Breast-fed infants are ensured a source of DHA and ARA until they are weaned from human milk. However, the need for a continued supply of preformed LCPs beyond weaning from breast-feeding is undetermined. The issue is further complicated by considerable variations in the duration of breast-feeding and levels of the LCPs in breast milk, which vary considerably, largely dependent on the maternal dietary intake.

Dietary supplementation of pre-formed LCPs in term infant formula continues to be controversial. See, SanGiovanni, J. P. et al., Early Human Development 2000; 57: 165-188. Several clinical trials have demonstrated that term infants receiving LCP-supplemented formula have more mature retinal function and cortical processing as measured by electroretinography (see, Birch, E. E. et al., Invest Ophthalmol Vis Sci 1996; 37: S1112), VEP acuity (see, Birch et al. (1998), opus cit.; see also, Hoffman et al. (2000), opus cit.; see also, Makrides, M. et al., Lancet 1995; 345:1463-1468) and preferential-looking acuity (see, Carlson, S. E. et al., Pediatr Res 1996; 39: 882-888). These reports were further supported by a meta-analysis of 12 published clinical trials; the authors concluded that term infants provided LCP-supplemented formula had more mature visual function than infants fed standard formulas. See, SanGiovanni et al. (2000), opus cit. Furthermore, cognitive development at both 10 (see, Willatts, P. et al., Lancet 1998; 352: 688-91) and 18 (see, Birch, E. E. et al., Dev Med Child Neurol 2000; 42: 174-181) months of age is associated with LCP-supplementation for the first 4 months of life. These studies, however, did not include infants that were breast-fed for part of their early maturation, up to the age of six months or later, and then weaned to supplemented formula. These infants' needs for continued DHA and ARA supplementation beyond weaning and until at least the age of one year went unrecognized.

In addition, several multi-center clinical trials have recently reported no benefit to either visual or cognitive development afforded by dietary LCP provision See, Auestad et al. (2001), opus cit.; see also, Lucas, A. et al., Lancet 1999; 354:1948-54; see also, Makrides, M. et al., Pediatrics 2000; 105:32-38. These differences between major studies may be attributable to variations in fatty acid levels, sources of LCPs, experimental design, or testing procedures. See, SanGiovanni et al. (2000), opus cit.; see also, Birch, E. E. et al. in: Handbook of Essential Fatty Acid Biology: Biochemistry, Physiology, and Behavioral Neurobiology (Yehuda et al., eds) Humana Press, Totowa, N.Y., pp. 183-199,1997; see also, Neuringer, M., Am J Clin Nutr 2000; 71:256S-267S; see also, Jensen, C. L. and Heird, W. C. Clin Perinatol 2002; 29:261-281. The need for LCPs earlier in the infant's diet may reflect the relatively rapid maturation of stereopsis in infancy.

Thus, there is a need to define the optimal duration for supplying LCP to the term infant, whether present in breast milk or in enriched formula. Many investigations considered that 2-to-4 months of breast-feeding or dietary LCP-supplementation would be sufficient to distinguish diet-related effects on blood lipid content and/or visual function (see, Birch et al. (1998), opus cit.; see also, Ponder, D. L. et al., Pediatr Res 1992; 32: 683-688; see also, Jorgensen et al., Lipids 1996; 31: 99-105; see also, Jensen, C. L. et al., J Pediatr 1997; 131: 200-209; see also, Innis, S. M. et al., Lipids 1997; 32: 63-72; see also, Innis S. M. et al., J Pediatr 2001; 139: 532-538.) More recently, infant nutritional trials have been extended for longer duration (see, Carlson et al. (1993), opus cit.; see also, Auestad et al. (2001); opus cit.; see also Lucas et al. (1999); opus cit.; see also Makrides et al. (2000), opus cit.), but these studies have focused on infants fed with supplemented and unsupplemented formula from birth. The need for DHA and ARA supplementation after weaning for those infants that were breast-fed during part of their early maturation period is still unrecognized.

It should be noted that these studies have employed measures of visual acuity not as early predictors of visual deficits that may later require ophthalmological care but rather as indices of the functional status of the brain. See Birch et al. (2000), opus cit. Thus, the underlying hypothesis is that quantification of subtle differences in visual acuity is an indirect measurement of differences in the maturation of brain function. See Id.

It has now been discovered that infants weaned from breast milk at 6 weeks derive visual function benefits when provided LCP-supplemented formula to one year of age. It has now also been discovered that term infants who were breast-fed until the age of 4 and 6 months or later have a continued need for DHA and ARA beyond that age to enhance early visual development. These breast-fed infants weaned at 4-6 months to LCP-supplemented formula still derived benefits at one year of age.

A need to provide LCPs in the infant's diet for at least 12 months is important not only with regards to breast-feeding infants but also to infants for which breast-feeding is not advised, possible or chosen. Some infants refuse to breast-feed or the mother is not physically able. In other instances, the mother may be infected with disease (e.g., human immunodeficiency virus) or may be under treatment with potent medications that may put the nurtured infant at risk. See Gartner et al. (1997), opus cit. In 1995, 59.4% of women in the United States breast-fed their infants at the time of hospital discharge and only 21.6% of mothers continued to nurse at 6 months. See Id. Thus, there is a need for a formula that provides the infant a balanced blood lipid fatty acid profile and optimizes visual function throughout the first year of life. The present invention indicates that such a formula should contain pre-formed DHA and ARA and be provided to breast-fed infants beyond weaning.

There is a present need for a method to enhance the visual development of term infants that have been breast-fed for only part of their early maturation period. The method must not negatively affect growth pattern, must be safe to be administered to infants, and, if administered as part of the nutritional intake of the infants, this feeding must be well tolerated by the infants.

### SUMMARY OF THE INVENTION

The present invention is directed to a novel method to enhance the visual development of term infants that are breast-fed for a number of months, including infants breast-fed up to the age of six months or later, and then weaned to formula. This novel method comprises administering the infants with a visual development enhancing amount of docosahexaenoic acid and arachidonic acid after the infants are weaned and continuing that administration for up to 1 year of age or later. The LCP fatty acids may be administered using a DHA- and ARA-supplemented formula. The formula does not negatively alter growth patterns, is well tolerated and imposes no safety issues.

### BRIEF DESCRIPTION OF THE DRAWINGS

FIG. 1 shows anthropometry of infant sub-groups at one-year of age weaned at 4 (n=16 and n=23) or 6 months (n=15 and n=7, respectively) to commercial formula (solid bars) or LCP-supplemented formula (striated bars).

FIG. 2 shows docosahexaenoic acid (DHA) levels, as relative percent, in red blood cell (RBC) lipids as function of age of breast-fed infants weaned at 4 or 6 months to commercial formula (solid squares) or LCP-supplemented formula (open squares).

FIG. 3 shows visual evoked potential (VEP) acuity in breast-fed infants weaned at 4 (A) or 6 (B) months as a function of age. Solid squares show infants weaned to commercial formula, and open squares show infants weaned to LCP-supplemented formula (open squares).

FIG. 4 shows stereoacuity in sub-sets of breast-fed infants weaned at 4 (A) or 6 (B) months of age. Solid squares show infants weaned to commercial formula, and open squares show infants weaned to LCP-supplemented formula.

FIG. 5 shows the association between 12-month sweep visual-evoked potential acuity values and red blood cell (RBC) levels of docosahexaenoic acid (DHA) in infants weaned at 4-6 months to commercial formula (solid squares) or LCP-supplemented formula (open squares).

FIG. 6 shows growth z scores for weight, length, and head circumference of infants weaned to LCP-supplemented formula or to control formula at 6 weeks of age.

FIG. 7 shows mean (+/−SEM) sweep visual evoked potential (VEP) acuity of infants weaned to LCP-supplemented formula or to control formula at 6 weeks of age.

FIG. 8 shows mean (+/−SEM) random dot stereoacuity of infants weaned to LCP-supplemented formula or to control formula at 6 weeks of age.

FIG. 9 shows sweep visual evoked potential (VEP) acuity of infants weaned at 6 weeks of age to LCP-supplemented formula or to control formula, and a comparison with a study on infants that were fed either LCP-supplemented formula or control formula since birth to the age of four months (Birch et al. (1998), opus cit)

### DETAILED DESCRIPTION OF THE INVENTION

The present invention relates to a method of enhancing the visual development of term infants who are breast-fed up to an age of from about one and one-half months to about six and one-half months and, then, weaned to formula. The method comprises administering to those infants a combination of DHA and ARA from the time they are weaned to formula. The length of time to administer the DHA/ARA combination to the infants is from at least one month to about one year. It is desirable that the combination of DHA and ARA be administered to the infants up to at least the age of one year.

In one embodiment of the invention, the combination of DHA and ARA is administered as part of an infant formula. The infant formula for use in the present invention is, typically, nutritionally complete and contains suitable types and amounts of lipids, carbohydrates, proteins, vitamins and minerals. The amount of lipids or fats typically can vary from about 3 to about 7 g/100 kcal. The amount of proteins typically can vary from about 1 to about 5 g/100 kcal. The amount of carbohydrates typically can vary from about 8 to about 14 g/100 kcal. Protein sources can be any used in the art, e.g., nonfat milk, whey protein, casein, soy protein, hydrolyzed protein, and amino acids. Lipid sources can be any used in the art, e.g., vegetable oils such as palm oil, soybean oil, palm olein oil, coconut oil, medium chain triglyceride oils, high oleic sunflower oil, and high oleic safflower oil. Carbohydrate sources can be any known in the art, e.g., lactose, glucose polymers, corn syrup solids, maltodextrins, sucrose, starch, and rice syrup solids. Conveniently, several commercially available infant formulas can be used. For example, Enfamile® with iron (available from Mead Johnson & Company, Evansville, Ind., U.S.A.) may be supplemented with suitable levels of ARA and DHA at the proper ratios and used to practice the method of the present invention. A particular infant formula suitable for use in the present invention is described in Example 3.

The form of administration of DHA and ARA in the method of the present invention is not critical, as long as a visual development enhancing amount is administered. Most conveniently, DHA and ARA are supplemented into an infant formula to be fed to the infants. Alternatively, DHA and ARA can be administered as a supplement not integral to formula feeding, for example, as oil drops, sachets or in combination with other nutrients such as vitamins.

The method of the invention requires a combination of DHA and ARA. The weight ratio of ARA:DHA is typically from about 1:3 to about 4:1. In one embodiment of the present invention, this ratio is from about 1:2 to about 3:1. In yet another embodiment, the ratio is from about 1:1 to about 2:1. In one particular embodiment the ratio is about 2:1.

The visual development enhancing amount of DHA for use in the present invention is typically from about 11 mg per kg of body weight per day to about 75 mg per kg of body weight per day. In one embodiment of the invention, the amount is from about 12 mg per kg of body weight per day to about 60 mg per kg of body weight per day. In another embodiment the amount is from about 13 mg per kg of body weight per day to about 50 mg per kg of body weight per day. In yet another embodiment the amount is from about 15 mg per kg of body weight per day to about 26 mg per kg of body weight per day.

The visual development enhancing amount of ARA for use in the present invention is typically from about 11 mg per kg of body weight per day to about 150 mg per kg of body weight per day. In one embodiment of this invention, the amount varies from about 12 mg per kg of body weight per day to about 120 mg per kg of body weight per day. In another embodiment, the amount varies from about 13 mg per kg of body weight per day to about 100 mg per kg of body weight per day. In yet another embodiment, the amount varies from about 15 mg per kg of body weight per day to about 52 mg per kg of body weight per day.

The amount of DHA in infant formulas for use in the present invention typically varies from about 12 mg/100 kcal to about 50 mg/100 kcal. In one embodiment of the present invention it varies from about 13 mg/100 kcal to about 33 mg/100 kcal; and in another embodiment from about 15 mg/100 kcal to about 20 mg/100 kcal. In a particular embodiment of the present invention, the amount of DHA is about 17 mg/100 kcal.

The amount of ARA in infant formulas for use in the present invention typically varies from about 12 mg/100 kcal to about 100 mg/100 kcal. In one embodiment of the present invention, the amount of ARA varies from about 13 mg/100 kcal to about 67 mg/100 kcal. In another embodiment the amount of ARA varies from about 15 mg/100 kcal to about 40 mg/100 kcal. In a particular embodiment of the present invention, the amount of ARA is about 34 mg/100 kcal.

The infant formula supplemented with oils containing DHA and ARA for use in the present invention can be made using standard techniques known in the art. For example, they can be added to the formula by replacing an equivalent amount of an oil, such as high oleic sunflower oil, normally present in the formula. As another example, the oils containing DHA and ARA can be added to the formula by replacing an equivalent amount of the rest of the overall fat blend normally present in the formula without DHA and ARA.

The source of DHA and ARA can be any source known in the art such as fish oil, single cell oil, egg yolk lipid, or brain lipid. In one embodiment of the present invention, sources of DHA and ARA are single cell oils as taught in U.S. Pat. Nos. 5,374,567; 5,550,156; and 5,397,591, the disclosures of which are incorporated herein in their entirety by reference. However, the present invention is not limited to only such oils. DHA and ARA can be in natural form provided that the remainder of the LCP source does not result in a deleterious effect on the infant. Alternatively, DHA and ARA can be used in refined form. The LCP source used in the present invention typically contain little or no EPA. For example, in one embodiment of the present invention the infant formula contains less than about 20 mg EPA/100 kcal; in another embodiment less than about 10 mg EPA/100 kcal; and in yet another embodiment less than about 5 mg EPA/100 kcal. One particular embodiment contains substantially no EPA. Another embodiment is free of EPA in that even trace amounts of EPA are absent from the formula.

### EXAMPLE 1

This example shows the results of a clinical study of breast-fed, term infants that were weaned at age 4 to 6 months to formula and which were then randomly assigned to a commercial formula with and without supplementation with DHA and ARA.

Participants/Eligibility:

Sixty-nine healthy term infants were recruited primarily from two hospitals in the north Dallas area, Presbyterian Medical Center and Medical City Columbia Hospital. All infants were born at 37 to 40 weeks postmenstrual age as determined by early sonograms, date of last menstrual period, and physical/neurodevelopmental assessment at birth. Only singleton births with birth weights appropriate for gestational age were included. Exclusion criteria were family history of milk-protein allergy, genetic or familial eye disease (e.g., hereditary retinal disease, strabismus), vegetarian or vegan maternal dietary patterns, maternal metabolic disease, anemia, or infection, presence of a congenital malformation or infection, jaundice, perinatal asphyxia, meconium aspiration, and any perinatal event which resulted in placement of the infant in neonatal intensive care.

Parents of eligible neonates were provided a brief information sheet about the study and were asked to call if they were planning to wean their infant from breast-feeding at 4 months of age. Parents were also informed that the American Academy of Pediatrics recommends breast-feeding for 12 months. All infants were breast-fed prior to weaning. No more than one formula feeding per day of a maximum 120 mL was permitted for inclusion in the trial. At 4 months, some mothers chose to continue breast-feeding. The eligibility criteria was extended to form a sub-group of infants weaned at 6 months of age (n=22); those who continued to breastfeed beyond 6 months were not included in the study.

Informed consent was obtained from one or both parents at the 1 ½-month appointment. The research protocol observed the tenets of the Declaration of Helsinki and was approved by the Institutional Review Boards of Presbyterian and Medical City Hospitals and the University of Texas Southwestern Medical Center (Dallas, Tex.).

Randomization:

Infants were enrolled at the 1 ½-month appointment (mean±SD=6.5±0.9 weeks of age) and randomized to receive one of two infant formulas at the time of weaning as described below. All infants were randomized using a single block randomization schedule at a central location; blocks were of variable lengths. The formula manufacturer (Mead-Johnson Nutritionals, Evansville, Ind.) masked both diets with two color/number codes for a total of 4 possible diet assignments for each infant.

Diets:

Study diets were a commercial infant formula (Enfamil® with iron, Mead Johnson) or the same commercial formula supplemented with 0.36% (of total fatty acids) DHA and 0.72% ARA. The mass and percent fatty acid composition of each formula is summarized in Table 1. The range of fatty acid values obtained from breast milk of a sub-set of study mothers (n=12) at 1 ½ months lactation are included for comparison purposes. This analysis utilized milk (15 ml) collected with medical grade breast pumps immediately after let-down. The commercial and LCP-enriched formulas provided approximately 15% of total fatty acids as LA and ˜1.5% as LNA yielding an overall ω6-to-ω3 ratio of 9.6 and 8.3, respectively, for the two formulas. The LCP-enriched formula contained single cell oils (DHASCO® and ARASCO®; Martek Biosciences, Columbia, Md.). Both formulas in 946 mL ready-to-feed cans provided 14.7 g/L protein, 37.5 g/L fat, 69.0 g/L carbohydrate, and 2805 kJ/L. All nutrients met existing standards for commercial formula established by the Infant Formula Act. Assigned diets were fed between 4 and 12 months of age for the 4-month weaning cohort and between 6 and 12 months for the 6-month weaning cohort. All participants' pediatricians recommended abstaining from solid foods before 4 months of age. Solid food intake following 4 months was not controlled.

TABLE 1
Fatty Acid Profiles of TERM Infant Study Diets

| | | |
|---|---|---|
| HM^{a} | CF^{b} | LCP-F^{b} |

| | |
|---|---|
| Individual Fatty Acids | % total fatty acid (g/L) |

| | | | |
|---|---|---|---|
| LA (18:2ω6) | 11.9-22.8 | 14.6 | 14.9 |
| | (4.0-9.7) | (8.5) | (8.4) |
| 20:3ω6 | 0.3-0.6 | 0 | 0.05 |
| | (0.01-0.35) | | (0.01) |
| ARA (20:4ω6) | 0.4-0.8 | 0 | 0.72 |
| | (0.15-0.46) | | (0.42) |
| LNA (18:3ω3) | 0.8-1.7 | 1.49 | 1.53 |
| | (0.28-0.74) | (0.86) | (0.86) |
| 20:5ω3 | 0.03-0.13 | 0 | 0 |
| | (0.02-0.05) | | |
| DHA (22:6ω3) | 0.07-0.38 | 0 | 0.36 |
| | (0.03-0.22) | | (0.21) |
| Totals | | | |
| C6-12 Saturates^{c} | 3.2-10.4 | 20.1 | 19.5 |
| | (1.1-5.8) | (11.7) | (10.9) |
| C14-24 Saturates^{d} | 29-45 | 34.4 | 33.6 |
| | (6.1-19.1) | (21.7) | (20.3) |
| Total | 28.1-47.9 | 29.4 | 29.2 |
| Monounsaturates^{e} | (7.8-27.2) | (17.1) | (16.4) |
| ω6/ω3 PUFA Ratio | 7.4-13.8 | 9.6 | 8.3 |

Notes:
Values given as % of total fatty acids and mass (g/L) in parenthesis;
^{a}HM = minimum and maximum human milk values from analysis of 12 samples collected at 47 ± 4 days lacation.
^{b}CF = commercial formula; LCP-F = formula supplemented with DHA and ARA; values given as mean from at least three determinations.
^{c}Includes 6:0, 8:0, 10:0, and 12:0.
^{d}Includes 14:0, 16:0, 17:0, 18:0, 20:0, 22:0 and 24:0.
^{e}Includes 14:1, 16:1, 18:1, 20:1, 22:1 and 24:1. Data for minor fatty acids (18:3ω6, 20:2ω6, 22:4ω6, 22:5ω3, 22:5ω6) not shown. LA = linoleic acid; ARA = arachidonic acid; LNA = α-linolenic acid; DHA = docosahexaenoic acid; PUFA are polyunsaturated fatty acids of ≧18 carbon chain length.

Experimental Design:

To meet eligibility criteria, infants needed to be weaned from breast-feeding at 4 or 6 months (±2 weeks). The general design for enrollment and follow-up testing are outlined in Table 2. In addition to weight and length at birth, infant growth measures included body weight, length, head circumference, triceps and subscapular fat folds at 1 ½, 4, 6, 9, and 12 months of age. Two blood samples were taken, one pre-weaning sample at either 4 or 6 months and one final outcome sample at 12 months. Visual acuity was determined by VEP at 1 ½, 4, 6, and 12 months in both groups and stereoacuity measurements were taken at 4, 6, 9, and 12 months. The testing and blood sampling protocols were selected to optimize testing of visual functions at time points that represent rapid maturation and to minimize maternal/infant stress associated with the blood draw. Parents and test examiners were masked to diet assignments.

Sample Size:

Sample sizes were estimated using the method described by Rosner. See Rosner B. Fundamentals of Biostatistics. Boston, Mass.; PWS-Kent Publishing Co., (1990), for α=0.05 and 1−β=0.90. Using standard deviations from our past studies of term infants for sweep VEP acuity [0.1 logMAR; i.e., one line on an eye chart], the final sample size per group at 12 months required to detect a 1 SD difference between groups is 21 infants. This sample size would also be sufficient to detect a 1 SD difference between groups in random dot stereoacuity [0.2 log see; e.g., 40 sec vs. 60 sec (16)] and a <1% difference in DHA or ARA fatty acid composition of RBCs.

Anticipating a 20-25% loss to follow-up over 12 months, a recruitment of 30 infants for each of the two diet groups was planned. The number of infants completing the study to 1 year reached 31 and 30 for the commercial formula and LCP-supplemented cohorts, respectively. However, the different weaning ages of two data sub-groups (4 vs. 6 months) provided unique information and is also reported. Sub-groups of infants weaned to commercial formula (n=16) or LCP-supplemented formula (n=23) at 4 months of age and sub-groups weaned at 6 months to commercial (n=15) or LCP-supplemented formula (n=7) were examined.

Presented in Table 2 is a summary of enrollment and the number of subjects who were tested at each time point and for whom complete data were available for analysis. Thirty-five infants in the commercial formula group and 34 infants in the LCP-supplemented group were enrolled at the 1 ½-month visit. The subsequent numbers of infants tested at each time point reflect those subjects that completed the full 12-month study and for which data were analyzed. Seven infants did not complete the study for reasons given below. In the commercial formula group, one subject attempted but was unable to wean from breast milk. For the other three subjects, the infants could not be scheduled for testing to meet protocol criteria. In the LCP-supplemented group, one infant was unable to wean, the parents of one infant could not be contacted for scheduling, and one infant discontinued after hospitalization for pneumonia (the child's pediatrician did not consider this associated with formula-feeding). The overall rate of loss to follow-up was 10.3%.

TABLE 2
Summary of Number of Infants at Entry and Included in Analyses
at Each Follow-up Testing Timepoint

| | Entry | | 1♯ months* | | 4 months* | | 6 months* | | 9 months* | | 12 months* | |
|---|---|---|---|---|---|---|---|---|---|---|---|---|
| | CF | LCP-F | CF | LCP-F | CF | LCP-F | CF | LCP-F | CF | LCP-F | CF | LCP-F |
| Growth | 35 | 33 | 31 | 30 | 31 | 30 | 31 | 30 | 31 | 30 | 31 | 30 |
| Blood Lipids** | — | — | — | — | 16 | 23 | 15 | 7 | — | — | 31 | 30 |
| VEP Acuity | — | — | 31 | 30 | 31 | 30 | 31 | 30 | — | — | 31 | 30 |
| Stereoacuity*** | — | — | — | — | 31 | 28 | 30 | 29 | 27 | 26 | 29 | 26 |

Notes:
Numbers of infants in commercial formula (CF) and long-chain polyunsaturated fatty acid-supplemented formula (LCP-F) groups are reported.
*Only infants completing all 5 visits were included in analyses (see text for details on 7 infants who discontinued prior to end of study).
**A baseline blood sample was drawn just prior to weaning, at either 4 or 6 months; an outcome blood sample was drawn for all participants at 12 months.
***Stereoacuity testing was not completed for all children on each visit (see text for details).

Growth:

Body weights were measured using a pediatric strain gauge (Healthometer, Bridgeview, Ill.) accurate to 1 g. Body lengths were measured using length boards (Ellard Instrumentation Ltd., Seattle, Wash.) accurate to 0.1 cm. Head circumference was measured using a non-stretching tape accurate to 0.1 cm. Subscapular and triceps fat deposition were measured using a Lafayette skin fold caliper (Lafayette Instruments, Lafayette, Ind.) accurate to 1 mm. Each length, head circumference, and skin fold measurement was made by two observers, and the average value recorded. Growth data of study infants were compared to published normative data released in 2000 by the Department of Health and Human Services as part of the National Health and Nutrition Examination Survey III (NHANES III).

Blood Lipid Fatty Acids:

Blood samples (2.0 mL) were collected into tubes (Microtainer®; Becton Dickinson, Franklin Lakes, N.J.) containing EDTA prior to weaning at either 4 or 6 months and again at 12 months via heel stick, aided by infant heel warming packs. Briefly, plasma and RBCs were separated by centrifugation, lipids extracted, transmethylated with boron trifluoride/methanol, and methyl esters analyzed by capillary column gas chromatography using flame ionization detection. Fatty acid peaks were identified by comparison to GLC68+11 standard and processed using custom semi-automated software. Fatty acids were quantified as percent of total fatty acids and also as mass concentrations (mg/L of plasma or packed RBC) based on the addition of internal standard (23:0 fatty acid). For fatty acid analysis of study formulas and breast milk samples, an extraction procedure was employed to limit loss of short-chain (6-12 carbon) fatty acids and methyl esters during sample processing. The analysis included extraction of lipids using 3% NH₄OH/100% ethanol/diethyl ether/petroleum hydrocarbons (2/2/5/5, by vol.) followed by two extractions with methylene chloride and a chromatographic temperature program modified to resolve short-chain fatty acid methyl esters.

Sweep VEP:

VEP acuity was assessed according to the sweep parameter protocol using vertical gratings phase-reversing at 6.6 Hz. Briefly, two bipolar placements of O_{z} vs. O₁ and O₂ were used to record (gain=10,000-20,000, −3 dB cut-off at 1 and 100 Hz) the electroencephalogram that was adaptively filtered in real time to isolate the VEP (397 Hz sampling rate). Amplitude and phase of the response at the second harmonic of the stimulation frequency was calculated for each channel. Noise was measured by determining the amplitude and phase of the two adjacent non-harmonic frequencies. Grating acuity was estimated with an automated algorithm which examines signal-to-noise ratio and phase coherence and performs a linear regression for the final descending limb of the vector averaged function (minimum of 3 trials, typically 5 trials) relating VEP second harmonic amplitude (amplitude at the reversal frequency of 13.2 Hz) to spatial frequency. Sweep VEP acuities were expressed in logMAR [log of the minimum angle of resolution; e.g., 20/20 (Snellen equivalent units) corresponds to a minimum angle of resolution of 1 min arc and logMAR of 0.0 while 20/200 corresponds to a minimum angle of resolution of 10 min arc and logMAR of 1.0].

Stereoacuity:

Random dot stereoacuity was assessed using Infant Random Dot Stereocards which utilizes a forced-choice preferential looking technique. Random dot stereoacuity was chosen as an outcome measure because it directly reflects cortical processing; detection of the disparate stimulus depends on cortical combination of monocular images that lack any form information. The Random Dot Stereocards consist of a series of test cards with disparities ranging from 1735″ to 45″ in approximate octave steps. The cards are presented in a 2-down-1-up staircase protocol. The infant views the test cards while wearing polarizing filters mounted in spectacle frames especially designed for infants and an observer judges each trial as whether the infant prefers to look at the disparate or the non-disparate stereogram. Stereoacuity is obtained by averaging (geometric mean) the last 6 of 8 reversals or by maximum likelihood estimation. To avoid bias introduced by “basement effects” in low vision eyes, criteria for switching over to the block method were established. Stereoacuity was expressed in log sec (log of the minimum detectable binocular disparity; e.g.,40 sec disparity corresponds to 1.60 log sec). As noted in Table 2, the stereoacuity test could not be completed on all infants on all visits. Of a total 244 testing visits at 4, 6, 9, and 12 months by all infants, 2 data points were unobtainable due to scheduling conflicts and 16 missed because the infants were too fussy and/or refused to wear the polaroid glasses necessary for testing.

Statistical Analysis:

All statistical comparisons were made between commercial formula and LCP-supplemented formula groups with infants weaned at 4 and 6 months combined. Where appropriate, sub-group analysis was conducted for infants weaned at 4 or 6 months of age. Data analysis for visual functions were conducted with repeated measures analysis of variance after verifying that the data met normality criteria. In cases where the interaction was significant, planned comparisons were conducted. Differences between anthropometric data of study groups were determined by Student's t-test. Statistical significance was set at p<0.05. For blood lipid fatty acids, more stringent criteria of significance (p<0.003) are reported as sixteen comparisons were made for each time point (Bonferroni adjustment=0.05/16=0.003). The association between blood fatty acids and visual outcomes was analyzed by linear regression and Pearson correlation analysis conducted to determine significance (p<0.05).

Summary of Results

Cohort Demographics:

Of the subjects that completed the 12-month trial (n=61), a majority were male (54%) and white (93%)(Table 3). There were no significant group differences (p>0.3) in maternal or paternal variables of age, body weight, or height. The number of both parents with maximum education at the high school level was greater (p<0.002 using Chi square analysis) in the LCP-supplemented group compared to the commercial formula group (38% vs 19%).

TABLE 3
Demographics of the Cohort*

| | Commercial Formula | LCP-Formula |
|---|---|---|
| N = | 31 | 30 |
| Gender (M/F) | 18/13 | 15/15 |
| White/Black/Hispanic/ | 29/0/1/1 | 28/0/2/0 |
| Asian | | |
| Maternal age (yr) | 31.3 ± 4.9 | 30.7 ± 4.7 |
| Maternal weight (kg) | 63.3 ± 14.4 | 59.9 ± 11.2 |
| Maternal height (cm) | 164 ± 7 | 165 ± 7 |
| Maternal education — | 19/68/13 | 38/45/17 |
| HS/college/postgrad (%) | | |
| Paternal age (yr) | 32.2 ± 4.4 | 32.2 ± 5.6 |
| Paternal weight (kg) | 85.8 ± 10.9 | 83.4 ± 14.0 |
| Paternal height (cm) | 181 ± 5 | 182 ± 7 |
| Paternal education — | 19/68/13 | 38/28/34 |
| HS/college/postgrad (%) | | |

Notes:
*lnfants were randomized by block design at entry; demographic data is based on data from infants completing the 12-month trial. Age and anthropometric data (mean ± SD) are not significantly different between groups (p > 0.3).

Growth:

Pre-weaning (4 months) and post-weaning (12 months) anthropometric measures of infant body weight, body length, and head circumference were not significantly different between the commercial and LCP-supplemented formula groups (p>0.3). Furthermore, z-scores were determined by comparison to national averages (NHANES III) and at 12 months were not different between groups for weight, length, weight-for-length, or head circumference (p=0.45, 0.44, 0.51, and 0.89, respectively). When the 4- and 6-month sub-groups were compared, no diet-related differences were found at one year of age for weight, length, head circumference, triceps or subscapular fat measures (FIG. 1).

Blood Lipid Fatty Acids:

The distribution of fatty acids in RBCs of infants prior to weaning at 4 or 6 months and at termination of the formula regime at 12 months of age is given in Table 4. Data are presented as relative percent of total fatty acids and as mass concentration (mg/L packed RBC; data in parenthesis).

TABLE 4
Fatty Acid Profiles in Total RBC Lipids of Study Infants^{a}

| | Pre-Weaning | | 12 Months | |
|---|---|---|---|---|
| | CF | LCP-F | CF | LCP-F |
| | N = 31 | n = 30 | n = 31 | n = 30 |
| | % total fatty acids (mg fatty acid/L RBC) | | | |
| ω3Fatty Acids | | | | |
| LNA | 0.145 ± 0.070 | 0.158 ± 0.063 | 0.193 ± 0.059 | 0.184 ± 0.070 |
| | (1.74) | (1.86) | (2.25) | (2.12) |
| 20:5ω3 | 0.277 ± 0.085 | 0.237 ± 0.042 | 0.247 ± 0.065 | 0.181 ± 0.050*^{¶} |
| | (2.84) | (2.78) | (2.86) | (2.05) |
| DPAω3 | 1.70 ± 0.30 | 1.65 ± 0.29 | 1.45 ± 0.19 | 0.84 ± 0.29*^{¶} |
| | (20.4) | (19.4) | (16.8) | (9.6) |
| DHA | 4.53 ± 0.82 | 4.50 ± 0.98 | 2.35 ± 0.48^{¶} | 5.88 ± 0.83*^{¶} |
| | (53.9) | (52.9) | (27.2) | (66.9) |
| ω6 Fatty Acids | | | | |
| LA | 12.5 ± 1.7 | 12.5 ± 1.4 | 14.6 ± 1.1^{¶} | 13.1 ± 1.0* |
| | (149) | (147) | (170) | (150) |
| 20:3ω6 | 1.54 ± 0.29 | 1.52 ± 0.24 | 1.67 ± 0.33 | 1.08 ± 0.22*^{¶} |
| | (18.2) | (17.8) | (19.4) | (12.3) |
| ARA | 15.2 ± 1.7 | 15.6 ± 1.2 | 14.3 ± 1.0 | 15.7 ± 0.9 |
| | (180) | (183) | (166) | (178) |
| 22:4ω6 | 3.86 ± 0.66 | 3.92 ± 0.53 | 4.10 ± 0.49 | 3.71 ± 0.31* |
| | (45.6) | (45.9) | (47.4) | (42.3) |
| DPAω6 | 1.07 ± 0.24 | 1.07 ± 0.19 | 1.05 ± 0.18 | 0.59 ± 0.17*^{¶} |
| | (12.6) | (12.5) | (12.2) | (6.75) |
| Totals | | | | |
| Total Saturates^{b} | 38.5 ± 1.3 | 38.3 ± 1.7 | 38.9 ± 0.9 | 38.6 ± 1.1 |
| | (458) | (449) | (451) | (440) |
| Total | 19.8 ± 2.2 | 19.6 ± 1.7 | 20.2 ± 1.4 | 19.4 ± 1.1 |
| Monounsaturates^{c} | (236) | (229) | (234) | (221) |
| Total ω3 LCP | 6.54 ± 0.96 | 6.41 ± 1.06 | 4.10 ± 0.60^{¶} | 6.92 ± 0.79* |
| | (78.1) | (75.3) | (47.3) | (78.7) |
| Total ω6 LCP | 22.1 ± 2.4 | 22.6 ± 1.6 | 21.7 ± 1.3 | 21.4 ± 1.2 |
| | (262) | (265) | (251) | (244) |
| Ratios | | | | |
| DHA/DPAω6 Ratio | 4.4 ± 1.4 | 4.4 ± 1.4 | 1.6 ± 0.3^{¶} | 7.7 ± 2.4*^{¶} |
| ω6/ω3 LCP Ratio | 3.4 ± 0.6 | 3.6 ± 0.6 | 5.4 ± 0.7^{¶} | 3.1 ± 0.4* |
| Unsaturation Index | 171 ± 8 | 172 ± 6 | 159 ± 5^{¶} | 172 ± 6* |
| | (2032) | (2015) | (1843) | (1960) |
| Notes: | | | | |
| ^{a}Values are mean ±1 SD for % of total fatty acids; mass data (mg fatty acid/L packed RBC given in parenthesis) for commercial formula-fed (CF) and long-chain polyunsaturated fatty acid supplemented formula (LCP-F) infant groups. | | | | |
| ^{b}Includes 14:0, 16:0, 17:0, 18:0, 20:0, 22:0 and 24:0. | | | | |
| ^{c}Includes 16:1, 18:1, 20:1, 22:1 and 24:1. LNA = α-linolenic acid, 18:3ω3; DPAω3 = docosapentaenoic acid, | | | | |
| # 22:5ω3; DHA = docosahexaenoic acid, 22:6ω3; LA = linoleic acid, 18:2ω6; ARA = arachidonic acid, 20:4ω6; | | | | |
| # DPAω6 = docosapentaenoic acid, 22:5ω6; LCP are >18 carbon chain length. Unsaturation Index is the sum of [# of double bonds x relative percent (or mass) of each fatty acid]. | | | | |
| # Supplemented group values in bold font | | | | |

^{¶} Values in bold at 12 months are significantly different from pre-weaning value at p < 0.003.

Prior to weaning, no significant differences between the two diet groups were found for any individual or summary fatty acid values. However, upon termination of the study diet period (at 12 months), highly significant differences between the commercial and LCP-supplemented formula groups emerged, reflecting incorporation of dietary DHA and ARA into RBC membrane lipids. RBC-DHA was 2.5-fold higher in the LCP-supplemented group. Dietary provision of the c LCP also resulted in significant, characteristic reductions (10% and 44%, respectively) of the ω6 LCPs 22:4ω6 and docosapentaenoic acid (DPAω6; 22:5ω6) compared to mean values of commercial formula-fed infants. A corresponding competition between ω3 and ω6 fatty acids for acyl incorporation into membrane phospholipids is reflected in reductions of eicosapentaenoic acid (20:5ω3) and docosapentaenoic acid (DPAω3; 22:5ω3) by 27% and 42%, respectively, were consistent with feeding of ω6 LCP and product inhibition due to DHA feeding. In the LCP-supplemented group, significant reductions of 10% and 35% in LA and dihomogamma-linolenic acid (20:3ω6), respectively, likely result from feed-back product inhibition of ω6 acyl incorporation into phospholipids due to the dietary supply of ARA. In contrast, no differences were found in individual (not shown) or total saturated or monounsaturated fatty acids between the two cohorts. Although the total sum of ω3 LCPs was elevated by 70%, there was no overall change in ω6 LCPs of the LCP-supplemented group at 12 months. This may be due to a more pronounced competition for incorporation into membrane phospholipids exerted by DHA compared to ARA. Furthermore, LCP supplementation resulted in marked elevations of the DHA/DPAω6 ratio, both end-products of ω3 and ω6 fatty acid metabolism. Despite changes in the dietary supply of many fatty acids introduced at the time of weaning, both DHA and ARA at 12 months of age in the LCP-supplemented group were maintained at levels equal to or greater than those in pre-weaning infants. In contrast, infants receiving commercial formula had a 50% reduction in DHA and a small but significant increase in LA levels at 12 months of age. This pattern in commercial formula-fed infants is also reflected by reductions in total ω3 LCPs, the DHA/DPAω6 ratio, and the unsaturation index. Feeding of commercial formula resulted in a marked elevation of the ω6/ω3 LCP ratio. After 6-to-8 months of ω3 and ω6 LCP supplementation, infants maintained indices of total ω3 and ω6 LCP levels as well as the overall unsaturated fatty acid nature of blood lipid membranes as reported by the unsaturation index. Mass analysis of RBC fatty acids reflected the same outcome.

Plotted in FIG. 2 are relative percent levels (mean±SE) of DHA in RBCs of infants in the 4- and 6-month sub-groups as a function of time on the study. Pre-weaning levels of RBC-DHA in breast-fed infants in the two diet groups were equivalent at both 4 and 6 months. By 12 months of age, the blood lipid level of DHA in infants receiving commercial formula dropped about 50% over the 6 to 8 month period while DHA levels in infants on the LCP-supplemented formula increased by 25 to 40%. In infants weaned at either 4 or 6 months of age, the mean DHA levels in RBC lipids of LCP-supplemented infants were 2.6- and 2.4-fold higher than in the commercial formula-fed group, respectively.

VEP Acuity:

Summary data for sweep VEP acuity measures are reported in Table 5 for the two randomized diet groups at the time of consent (1 ½ months), pre-weaning (4 months) and at termination of study diets (12 months). A repeat measures ANOVA revealed a significant interaction in VEP acuity (p=0.0009) as well as significant main effects of age (p<0.0005) and of diet (p<0.0005). In the planned comparisons, there were no significant differences at 1 ½ or 4 months (p>0.48) as all infants were receiving breast milk. At 12-month, the LCP-supplemented infants had significantly (p<0.0005) better VEP acuity than infants in the commercial formula group by about 0.1 logMAR or about 1 line on an eye chart. The results were also examined for the two sub-sets of infants weaning at either 4 or 6 months of age. FIG. 3 graphically displays progression of visual acuity maturation in the 4-month weaning cohort (3 A.) and in the 6-month cohort (3 B.). Lower numeric values of Snellen equivalents correspond to better, more mature visual acuity. In both sub-sets, the LCP-supplemented groups had significantly better acuity than in commercial formula-fed groups at 12 months, p=0.01 and p=0.03, respectively. However, there was also a significant (p=0.03) benefit of LCP-supplementation found at the 6-month time point in infants weaned to formula at 4 months of age. Thus, during a 2-month period, LCP supplementation was sufficient to modify visual function in breast-fed term infants.

TABLE 5
Visual Evoked Potential (VEP) Acuity and Random Dot
Stereoacuity in Study Infants^{a}
Visual Evoked Potential Acuity

| | n = | 1½ Months | 4 Months | 12 Months |
|---|---|---|---|---|
| CF | 31 | 0.742 ± 0.112 | 0.476 ± 0.115 | 0.255 ± 0.128 |
| | | (0.020) | (0.021) | (0.023) |
| LCP-F | 30 | 0.724 ± 0.103 | 0.515 ± 0.113 | 0.152 ± 0.080* |
| | | (0.019) | (0.021) | (0.015) |

Random Dot Stereoacuity

| | | | | | | |
|---|---|---|---|---|---|---|
| | n = | 4 Months | n = | 9 Months | n = | 12 Months |
| CF | 31 | 2.761 ± 0.635 | 27 | 2.215 ± 0.616 | 29 | 2.143 ± 0.553 |
| | | (0.114) | | (0.118) | | (0.103) |
| LCP-F | 28 | 2.850 ± 0.544 | 26 | 2.013 ± 0.287 | 26 | 1.965 ± 0.268 |
| | | (0.103) | | (0.056) | | (0.052) |

Notes:
^{a}Values are mean ± SD (with SE in parenthesis) for commercial formula (CF) and long-chain polyunsaturate-supplemented formula (LCP-F) groups. Visual-evoked potential (VEP) acuity values are reported as logMAR and random dot stereoacuity values are reported as log sec.
*Values in bold font are significantly different from CF at p < 0.0005.

Stereoacuity:

Summary data for Random Dot Stereoacuity measures in the two diet groups are given in Table 5. For infants weaned at either 4 or 6 months, there were significant main effects of age (p<0.0005); however, there were no significant diet-related differences in stereoacuity. As shown in FIG. 4A and B, there was a trend for improved stereoacuity (i.e., lower numeric values) in the two LCP-supplemented sub-groups.

Correlations between Visual Function and RBC Fatty Acids:

The relationship between sweep VEP acuity and the relative percent levels of DHA in RBCs was examined in 12-month old infants by linear regression analysis (FIG. 5). A highly significant correlation was found (r=−0.42; r²=0.18; p<0.0005) such that infants with higher contents of RBC-DHA had more mature visual cortical function. This data also indicates that RBC-DHA alone accounts for 18% of the variability in sweep VEP acuity in study infants at 12 months of age. VEP acuity was also significantly correlated with the sum of ω3LCPs (r=−0.44; p=0.0004), and with the DHA/DPAω6 ratio (r=-0.34; p=0.006). In addition, the unsaturation index was also correlated with VEP acuity (r=−0.04; p=0.001) such that a more highly unsaturated membrane corresponded with better acuity. In contrast, the ω6LCP/ω3LCP ratio was positively correlated with VEP acuity (r=0.38; p=0.002) meaning that higher levels of ω16LCPs relative to ω3LCPs were associated with poorer visual acuity. Similarly, positive correlations were also found with LA (r=0.35; p=0.005), ARA (r=0.38; p=0.002) and oleic acid (18:1ω9; r=0.38; p=0.002) in 12-month old infants.

Although stereoacuity was not correlated with ω3 or ω6 fatty acids, it was modestly associated with the polyunsaturated nature of RBC membranes. Stereoacuity was negatively correlated with the unsaturation index (r=−0.33; p=0.01) and the ratio of polyunsaturated-to-saturated fatty acids (r=−0.32; p=0.02) such that membranes containing more unsaturated fatty acids were associated with better stereoacuity in infants at 12 months of age.

Brief Discussion of Results:

The present invention, as demonstrated by the results of this randomized clinical trial, shows the benefits of supplying DHA and ARA in an infant's diet beyond 4 to 6 months of age in the optimization of the infant's early visual development. The formula supplemented with DHA and ARA from single cell oil sources did not alter growth patterns, was well tolerated, and imposed no safety issues.

One of the most unexpected results of this trial is the benefit seen in visual function at the 6-month time point of the cohort weaned at 4-months (FIG. 3A). This study shows a statistically significant improvement in VEP acuity in the LCP-supplemented group compared to the commercial formula-fed group occurring over a relatively short 2-month period and, thus, it suggests that a supply of pre-formed DHA may be of critical importance during this 2-month period. The current results provide evidence that supplementation of pre-formed LCPs in the post-weaning diet is beneficial for functional development during the first year of life. Furthermore, a post-weaning supply of LCPs was found to sustain DHA blood lipid levels from that of a breast-fed infant at weaning, either at 4 or 6 months, out to 12 months of age. In this trial, a commercial formula with a recommended ratio of the dietary essential fatty acids, LA:LNA of 10:1 resulted in a 50% decrease in blood lipid DHA content and 6-10% loss in ARA after a 6- to 8-month commercial formula regime. Provision of this formula enriched with 0.36% DHA and 0.72% ARA not only maintains these LCP levels in blood lipids but also optimizes visual functional maturation in infancy.

DHA and ARA levels in the LCP-supplemented formula fall within the concentration ranges (mass and percentage) found in breast milk samples obtained from a sub-set of study mothers (Table 1). These values are consistent with those of women consuming Western diets.

There was a small but significant elevation in the unsaturation index of LCP-supplemented infants compared to commercial formula-fed infants at 1 year (Table 4); this was due to a significant decrease in fatty acid unsaturation in the commercial formula group. Fatty acid unsaturation, reflecting the overall content of double bonds in RBC membrane lipids, was also correlated with both VEP acuity and stereoacuity at 12 months of age in the whole cohort.

Both VEP acuity and stereoacuity are dependent initially on proper retinal maturation as well as neural processing in the visual cortex. The degree of fatty acid unsaturation may influence the function of various membrane-related enzymes, receptors, and nutrient transport systems and, thus, impact retinal and cortical transduction of visual stimuli throughout infant development.

In conclusion, the study indicates that the present invention fulfills the need for a formula that provides a term infant with a balanced blood lipid fatty acid profile and optimizes visual development throughout the first year of life. The present invention provides that a formula containing pre-formed DHA and ARA be provided to breast-fed infants beyond weaning at 4 or 6 months of age.

### EXAMPLE 2

This example shows the results of a clinical study of breast-fed term infants that were weaned at the age of six weeks to formula and who were randomly assigned to a commercial formula with and without supplementation with ARA and DHA. The assigned diets were maintained until the infants reached the age of 1 year.

Subiects:

Sixty-five healthy term infants born in the Dallas area were enrolled in the randomized clinical trial at 6 weeks of age. All participants were born at 37-40 weeks postmenstrual age as determined by an early sonogram, the date of the last menstrual period, and physical and neurodevelopmental assessment at birth. Only singleton births with birth weights appropriate for gestational age were included. Exclusion criteria were family history of milk protein allergy; genetic or familial eye disease (eg, hereditary retinal disease, strabismus); vegetarian or vegan maternal dietary patterns; maternal metabolic disease, anemia, or infection; presence of a congenital malformation or infection; jaundice; perinatal asphyxia; meconium aspiration; and any perinatal event that resulted in placement of the infant in the neonatal intensive care unit.

Parents of eligible neonates were provided a brief information sheet about the study and were asked to call if they were planning to wean the infant from breastfeeding at 6 weeks of age. Parents also were informed that the American Academy of Pediatrics recommends breastfeeding for 12 months and that other ongoing studies in the laboratory were available for infants who are breastfed for more than 6 weeks. Informed consent was obtained from one or both parents at the 6-weeks appointment, before the infant's participation. This research protocol observed the tenets of the Declaration of Helsinki and was approved by the Institutional Review Boards of the University of Texas Southwestern Medical Center (Dallas), Presbyterian Medical Center (Dallas), and Medical City Columbia Hospital (Dallas).

Randomization:

Infants were randomly assigned on the day of enrollment (target age of 6 weeks; range: 4-8 weeks; mean±SD age: 5.1±1.2 weeks) to consume 1 of the 2 diets described in the paragraph below. Most families were recruited from 2 separate hospitals to encourage ethnic and socioeconomic diversity in the cohort; a few infants were recruited at other sites when their parents learned about the study from friends or relatives and contacted us. All infants were randomly assigned with the use of a single randomization schedule at a central location. Both diets were masked by 2 color and 2 number codes, for a total of 4 possible diet assignments for each infant. Diet assignments, based on a blocked randomization schedule (with variable length blocks), were provided in sealed envelopes to the study site.

Diets:

The study diets were commercial infant formula (Enfamil with iron; Mead Johnson Nutritional Group, Evansville, Ind.) or the same commercial infant formula supplemented with 0.36% of total fatty acids as docosahexaenoic acid (DHA; 22:6n3) and 0.72% as arachidonic acid (ARA; 20:4n6). The fatty acid composition of both formulas and of human milk is summarized in Table 6. Both formulas provided 15% linoleic acid (LA; 18:2n6) and 1.5% linolenic acid (LNA; 18:3n3). The LCP (DHA+ARA) supplemented formula contained single cell oils (DHASCO and ARASCO; Martek Biosciences, Columbia, Md.). Both formulas were provided in 946-mL ready-to-feed cans and provided 14.7 g protein/L, 37.5 g fat/L, 69.0 g carbohydrate/L, and 2805 kJ/L. All nutrients met existing standards for commercial formula established by the Infant Formula Act. Assigned diets were fed between 6 and 52 weeks of age. None of the infants had solid food before 17 weeks of age, and most infants had no solid food other than cereal until 26 weeks of age.

TABLE 6
Fatty acid profiles of term infant study formulas¹

| | HM | CF | LCP |
|---|---|---|---|
| Individual | g/L (% total fatty acids) | | |
| fatty acids | | | |
| 18:2n−6 | 7.16 ± 4.02² (16.0) | 8.48 (14.6) | 8.37 (14.9) |
| 20:3n−6 | 0.20 ± 0.09 (0.5) | 0 | 0.01 (0.05) |
| 20:4n−6 | 0.27 ± 0.13 (0.61) | 0 | 0.42 (0.72) |
| 18:3n−3 | 0.55 ± 0.21 (1.28) | 0.86 (1.49) | 0.86 (1.53) |
| 20:5n−3 | 0.03 ± 0.01 (0.07) | 0 | 0 |
| 22:6n−3 | 0.12 ± 0.04 (0.19) | 0 | 0.21 (0.36) |
| Totals | | | |
| C₆₋₁₂ SFA ³ | 3.8 ± 3.2 (7.7) | 11.67 (20.1) | 10.97 (19.5) |
| C₁₄₋₂₄ SFA ⁴ | 14.9 ± 5.5 (34.0) | 21.69 (34.4) | 20.36 (33.6) |
| Total MUFA ⁵ | 15.8 ± 6.7 (38.4) | 17.11 (29.4) | 16.42 (29.2) |
| Ratios | | | |
| P:S | 0.46 ± 0.12 | 0.30 | 0.33 |
| n−6:n−3 PUFA | 9.8 ± 1.7 | 9.6 | 8.3 |

Notes:
¹ mean of ≧3 determinations. HM, human milk; CF, commercial formula; LCP, formula supplemented with docosahexaenoic acid and arachidonic acid; SFA, saturated fatty acids; MUFA, monounsaturated fatty acids; P:S, ratio of polyunsaturated to saturated fatty acids; PUFA, polyunsaturated fatty acids of ≧ 18-carbon chain length.
² mean ± SD from analysis of 12 samples collected at 47 ± 4 d lactation.
³ Includes 6:0, 8:0, 10:0, and 12:0.
⁴ Includes 14:0, 16:0, 17:0, 18:0, 20:0, 22:0, and 24:0.
⁵ Includes 14:1, 16:1, 18:1, 20:1, 22:1, and 24:1. Data for minor fatty acids (18:3n−6, 20:2n−6, 22:4n−6, 22:5n−3, and 22:5n−6) are not shown.

General Protocol:

Before 6 weeks of age, one feeding of commercial formula per day was permitted (maximum of 120 mL at a single feeding). After randomization at the 6-weeks appointment, complete weaning from breastfeeding to formula feeding had to be accomplished within 2 weeks. Examiners who were blinded to diet assignment conducted all tests.

Sweep acuity, as measured by cortical visual evoked potentials (VEPs), and growth were measured at 6, 17, 26, and 52 weeks. The 6-weeks time point provided a baseline measurement at the time of randomization. The 17 and 26-weeks time points were included because they allow for maximum exposure to LCP supplementation (because little or no solid food was given to infants before 17 or 26 weeks) and because sweep VEP acuity normally develops rapidly during that time. The 52-weeks time point was included because it represents the maximum length of exposure to LCP-supplemented formula and because sweep VEP acuity is relatively mature at this time point [0.3 log of the minimum angle of resolution (log MAR) below the adult value].

Stereoacuity was assessed at 17, 26, 39, and 52 weeks of age. The 6-weeks time point was excluded because less than 5% of infants would be expected to demonstrate stereoacuity at this early age. The 17, 26, and 52-weeks time points correspond to those for VEP and growth measurements, and the 39-weeks time point was added to provide more detailed information for assessment of the rate of maturation because this outcome variable had not been used previously in a randomized clinical trial of infant nutrition.

Sample Size:

Sample sizes were estimated by using the method described by Rosner, opus cit. for α=0.05 and 1−β=0.90. With the use of standard deviations for sweep VEP (0.1 log MAR; ie, one line on an eye chart) from our present and past studies of term infants, the final sample size per group at 12 months required to detect a 1-SD difference between groups is 21 infants. This sample size will also be sufficient to detect a 1-SD difference between groups in random dot stereoacuity (0.2 log s; e.g., 40 s compared with 60 s) and a <1% difference in the DHA or ARA fatty acid composition of red blood cells (RBCs). Anticipating a 20-25% loss to follow-up over 12 months, the recruitment of 30 infants for each of the 2 diet groups was planned; the achieved enrollment was of 32 and 33 per group.

TABLE 7
Summary of enrollment, loss to follow-up, and testing schedule
(Weaning at 6 weeks of age study)

| | 6 weeks | | 17 weeks | | 26 weeks | | 39 weeks | | 52 weeks | |
|---|---|---|---|---|---|---|---|---|---|---|
| | LCP | Control | LCP | Control | LCP | Control | LCP | Control | LCP | Control |
| Enrollment | 32 | 33 | N/A | N/A | N/A | N/A | N/A | N/A | N/A | N/A |
| Growth | 32 | 33 | 29 | 31 | 28 | 32 | N/A | N/A | 28 | 30 |
| Blood Lipids | N/A | N/A | 28 | 31 | N/A | N/A | N/A | N/A | 28 | 30 |
| VEP Acuity | 32 | 33 | 29 | 31 | 29 | 31 | N/A | N/A | 28 | 30 |
| Stereoacuity* | N/A | N/A | 28 | 30 | 26 | 31 | 27 | 27 | 25 | 28 |

Notes:
*Stereoacuity testing was not completed for all children on each visit (see text for details).

A summary of enrollment and loss to follow-up is presented in Table 7. Seven infants (10.7%) were lost to follow-up during the course of the study. Of those 7, 5 infants (7.6%) dropped out of the study after the initial appointment at 6 weeks. In 3 cases, the infants were withdrawn from the study because of their pediatricians' recommendation to switch to a soy-protein-based formula after the infants had symptoms suggestive of intolerance to lactose or cow milk protein. In one case, the mother was unable to wean the infant to formula, and in another case the parent could not be contacted to schedule a visit. Of the 60 infants who remained in the study after randomization at 6 weeks of age, 58 (96.7%) completed the protocol through 12 months of age. Two children dropped out of the study after the 26-weeks visit: one because of asthma possibly related to milk allergy and one because the parent could not be contacted to schedule a visit. Sample sizes at 12 months were 28 in the LCP-supplemented-formula group and 30 in the control-formula group.

Sweep VEP acuity:

VEP acuity was assessed according to the swept parameter protocol developed by Norcia and colleagues (Norcia et al., Vision Res 1985; 25:1399-408) with the use of vertical-gratings phase reversing at 6.6 Hz. Briefly, 2 active electrodes (O₁ and O₂) referenced against an electrode at O_{z} were used to record (gain: 10000-20000, 3-decibel cutoff at 1 and 100 Hz) the electroencephalogram that was adaptively filtered in real time to isolate the VEP (397-Hz sampling rate). The amplitude and phase of the response at the second harmonic of the stimulation frequency were calculated for each channel. Noise was measured by determining the amplitude and phase of the 2 adjacent nonharmonic frequencies. Grating acuity was estimated with an auto-mated algorithm that examines signal-to-noise ratio and phase coherence and performs a linear regression for the final descending limb of the vector-averaged function (minimum of 3 trials; typically 5 trials) relating VEP second-harmonic amplitude (amplitude at the reversal frequency of 13.2 Hz) to spatial frequency. Sweep VEP acuities were expressed in log MAR (e.g., 20/20 corresponds to an MAR of 1 min arc and log MAR of 0.0 whereas 20/200 corresponds to an MAR of 10 min arc and log MAR of 1.0).

Stereoacuity:

Random dot stereoacuity was assessed with the use of forced-choice preferential looking and the infant random dot stereo-cards. Random dot stereoacuity was chosen as an outcome measure because it directly reflects cortical processing; detection of the disparate stimulus depends on the cortical combination of monocular images that lack any form information. The random dot stereocards consist of a series of test cards with disparities ranging from 1735 to 45 s arc in approximate octave steps. The cards are presented in a 2-down, 1-up staircase protocol. The infant views the test cards while wearing polarizing filters mounted in spectacle frames especially designed for infants, and an observer judges on each trial whether the infant prefers to look at a disparate or a nondisparate stereogram. Stereoacuity is obtained by averaging (geometric mean) the last 6 of 8 reversals or by maximum likelihood estimation. To avoid bias introduced by basement effects in low-vision eyes, it was established criteria for switching over to the block method. Stereoacuity was expressed in log s (log of the minimum detectable binocular disparity; e.g., a 40-s disparity corresponds to 1.60 log s). As noted in Table 7, the stereoacuity test could not be completed on all infants at all visits because the polarized glasses required could not be placed on the child because of conjunctivitis (1 child in the LCP-supplemented-formula group at 26 weeks and 1 child in the control-formula group at 39 weeks), because the child refused to wear the glasses (1 child in the LCP-supplemented-formula group at 26 weeks, 1 child in the LCP-supplemented-formula group and 2 children in the control-formula group at 39 weeks, and 3 children in the LCP-supplemented-formula group and 2 children in the control-formula group at 52 weeks), or because the child had a tropia at the time of testing (1 child in the LCP-supplemented-formula group and 1 child in the control-formula group at 17 weeks and 1 child in the LCP-supplemented-formula group at 26 weeks).

Growth:

Weight was measured by using a pediatric strain gauge scale (Healthometer, Bridgeview, Ill.) accurate to 1 g. Length was measured by using length boards (Ellard Instrumentation Ltd, Seattle) accurate to 0.1 cm. Growth data were normalized by expressing them as z scores for term infants of the appropriate age and sex and by using the LMS parameters provided in the data files from the Centers for Disease Control and Prevention (CDC) growth charts released in 2000 by the Department of Health and Human Services as part of the National Health and Nutrition Examination Survey.

Blood Lipids:

Blood samples (2.0 mL) were collected at 17 and 52 weeks by heel stick aided by infant heel warming packs into tubes (Microtainer; Becton Dickinson, Franklin Lakes, N.J.) containing EDTA. Plasma and RBCs were separated by centrifugation at 3000 g for 10 min at 4C, lipids were extracted and transmethylated with boron trifluoride-methanol, and methylesters were analyzed by capillary gas chromatography with flame ionization detection. Fatty acid peaks were identified by comparison with the GLC68+11 standard and by using custom software that semi-automated data processing. Concentrations were obtained as mass concentrations (mg/L plasma or packed RBCs) on the basis of the addition of an internal standard (23:0).

Statistical Analyses:

During the course of the study, all data were handled in a coded manner. The data were analyzed with two-way repeated-measures analysis of variance after verifying that they met normality criteria. Planned comparisons were carried out to compare the means of the 2 diet groups at each age point. Because 4 pair-wise comparisons were conducted for each of the vision outcome variables (acuity and stereoacuity), only planned comparisons with P<0.01 were considered

significant (Bonferroni adjustment of 0.05/4, or 0.0125). Linear regression was conducted to examine the association between blood lipid concentrations and visual outcomes. Because linear regression was conducted to examine the relation between 4 major fatty acids (LA, A-LNA, ARA, and DHA), only regression coefficients associated with P<0.01 were considered significant (Bonferroni adjustment of 0.05/4, or 0.0125).

Summary of Results

Demographics of the cohort:

Ethnic representation in the cohort was similar to that of the greater Dallas area: 77% white, 23% minority. Sixty-one percent of the cohort was male and 39% was female. Maternal variables included a mean age of 30 y, mean pre-pregnancy weight of 64 kg, and mean height of 1.66 m. Paternal variables included a mean age of 32 y, mean weight of 86 kg, and mean height of 1.81 m. Sixty-nine percent of mothers and 75% of fathers had completed at least 2 y of college education. Demographic information for the individual diet groups is summarized in Table 8. There were no significant differences between the groups in recruitment site, sex representation, ethnicity, or maternal and paternal variables assessed.

TABLE 8
Demographics of the Cohort

| | LCP-Supplemented | Control |
|---|---|---|
| N | 32 | 33 |
| Recruitment from | 18/8/6 | 20/7/6 |
| Hospital 1/Hospital 2/Other | | |
| Gender (M/F) | 19/13 | 21/12 |
| % White/% Minority | 75%/25% | 78%/22% |
| Maternal Age (yr) | 29.5 ± 4.7 | 30.7 ± 4.1 |
| Maternal Weight (kg) | 63.7 ± 14.2 | 65.0 ± 9.8 |
| Maternal Height (m) | 1.65 ± 0.07 | 1.68 ± 0.08 |
| Paternal Age (yr) | 32.1 ± 4.8 | 32.5 ± 4.6 |
| Paternal Weight (kg) | 87.5 ± 13.8 | 85.2 ± 8.9 |
| Paternal Height (m) | 1.82 ± 0.08 | 1.81 ± 0.07 |
| Maternal Education | | |
| (% high school/% college/ | 37.5%/53%/9.5% | 24%/67%/9% |
| % post-graduate) | | |
| Paternal Education | 31%/44%/25% | 18%/55%/27% |
| (% high school/% college/ | | |
| % post-graduate) | | |

Blood Lipids:

The mean concentrations of major fatty acids in plasma and RBC total lipids for both randomized diet groups at 17 and 52 weeks of age are summarized in Tables 9 and 10, respectively. At 17 weeks of age, both plasma and RBC concentrations of DHA were significantly higher in infants who consumed LCP-supplemented formula than in those who consumed control formula. At 52 weeks, plasma and RBC concentrations of DHA were similar to those at 17 weeks; ie, plasma and RBC concentrations of DHA were significantly higher in infants who consumed LCP-supplemented formula than in those who consumed control formula. Moreover, there was an even greater difference between the 2 diet groups in the RBC concentrations of DHA at 52 weeks than at 17 weeks. There were no significant differences in the concentrations of A-LNA or eicosapentaenoic acid in plasma at either age, but n3 docosapentaenoic acid (n3 DPA; 22:5n3) was significantly lower in the LCP-supplemented-formula group than in the control-formula group at both ages. In RBC lipids, there were no significant differences in α-LNA concentrations, whereas eicosapentaenoic acid and n3 DPA were lower in the LCP-supplemented-formula group than in the control-formula group at both 17 and 52 weeks.

TABLE 9
Fatty acid profiles in total plasma lipids of study infants¹

| | | | 52 weeks | |
|---|---|---|---|---|
| | 17 weeks | | | LCP- |
| | | LCP- | Control- | supplemented- |
| | Control- | supplemented- | formula | formula |
| | formula group | formula group | group | group |
| | (n = 31) | (n = 29) | (n = 30) | (n = 28) |
| n6 Fatty acids | | | | |
| 18:2n−6 | 351 ± 54 | 229 ± 40² | 347 ± 56 | 348 ± 26 |
| (mg/L) | | | | |
| 20:3n−6 | 17.6 ± 3.6 | 12.5 ± 3.6² | 21.6 ± 5.0 | 15.0 ± 5.8² |
| (mg/L) | | | | |
| 20:4n−6 | 61.4 ± 11.0 | 109 ± 19² | 75.5 ± 14.7 | 109 ± 21² |
| (mg/L) | | | | |
| 22:4n−6 | 3.71 ± 0.69 | 3.68 ± 0.63 | 4.94 ± 1.20 | 4.91 ± 1.58 |
| (mg/L) | | | | |
| 22:5n−6 | .77 ± 1.10 | 2.09 ± 0.61² | 4.80 ± 0.97 | 2.52 ± 1.00² |
| (mg/L) | | | | |
| n3 Fatty acids | | | | |
| 18:3n−3 | 8.56 ± 2.57 | 7.56 ± 1.98 | 7.31 ± 2.91 | 8.57 ± 4.11 |
| (mg/L) | | | | |
| 20:5n−3 | 1.62 ± 0.43 | 1.36 ± 0.45 | 2.60 ± 0.92 | 2.02 ± 1.1⁵ |
| (mg/L) | | | | |
| 22:5n−3 | 4.43 ± 1.04 | 2.28 ± 0.61² | 5.95 ± 1.07 | 3.33 ± 1.21² |
| (mg/L) | | | | |
| 22:6n−3 | 14.5 ± 3.1 | 42.7 ± 7.0² | 13.1 ± 3.4 | 43.3 ± 9.0² |
| (mg/L) | | | | |
| Totals | | | | |
| Total SFA | 478 ± 96 | 451 ± 60 | 440 ± 74 | 499 ± 103 |
| (mg/L)³ | | | | |
| Total MUFA | 331 ± 81 | 278 ± 51 | 308 ± 70 | 341 ± 93⁵ |
| (mg/L)⁴ | | | | |
| Total n−6 LCP | 90.6 ± 13.9 | 131 ± 21² | 110 ± 17 | 135 ± 22² |
| (mg/L) | | | | |
| Total n−3 LCP | 20.8 ± 4.1 | 46.6 ± 7.4² | 21.8 ± 4.3 | 48.8 ± 8.7² |
| (mg/L) | | | | |
| Ratios | | | | |
| DHA:n−6 DPA | 4.16 ± 1.67 | 21.7 ± 5.6² | 2.78 ± 0.61 | 19.5 ± 7.1² |
| n−6:n−3 LCP | 4.41 ± 0.48 | 2.84 ± 0.41² | 5.12 ± 0.53 | 2.79 ± 0.32² |
| Mead | 0.019 ± 0.008 | 0.005 ± 0.004² | 0.019 ± 0.006 | 0.009 ± 0.006² |
| acid:ARA | | | | |
| Unsaturation | 1540 ± 245 | 1700 ± 223⁵ | 1590 ± 197 | 1890 ± 312⁵ |
| index | | | | |

Notes:
¹mean ± SD. LCP, long-chain polyunsaturated fatty acids; AA, arachidonic acid; DPA, docosapentaenoic acid; DHA, docosahexaenoic acid; Mead acid, 20:3n9. The unsaturation index is the sum of the number of double bonds in each fatty acid the mass of each fatty acid for all the fatty acids.
^{2,5}Significantly different from the control-formula group: ²P < 0.001, ⁵P < 0.01.
³Includes 14:0, 16:0, 17:0, 18:0, 20:0, 22:0, and 24:0.
⁴Includes 14:1, 16:1, 18:1, 20:1, 22:1, and 24:1.

TABLE 10
Fatty acid profiles in total red blood cell lipids of
study infants¹

| | 17 weeks | | 52 weeks | |
|---|---|---|---|---|
| | | LCP- | | LCP- |
| | Control- | supplemented- | Control- | supplemented- |
| | formula group | formula | formula group | formula |
| | (n = 31) | group (n = 29) | (n = 30) | group (n = 28) |
| n6 Fatty acids | | | | |
| 18:2n−6 | 155 ± 25 | 126 ± 19² | 157 ± 21 | 149 ± 33 |
| (mg/L) | | | | |
| 20:3n−6 | 19.5 ± 5.7 | 13.7 ± 3.0² | 20.2 ± 4.9 | 12.5 ± 3.8² |
| (mg/L) | | | | |
| 20:4n−6 | 159 ± 25 | 178 ± 20³ | 157 ± 21 | 174 ± 26 |
| (mg/L) | | | | |
| 22:4n−6 | 45.2 ± 5.8 | 40.1 ± 7.0³ | 46.1 ± 6.5 | 40.3 ± 5.8³ |
| (mg/L) | | | | |
| 22:5n−6 | 12.7 ± 2.0 | 8.55 ± 1.93² | 11.7 ± 2.2 | 6.17 ± 1.78² |
| (mg/L) | | | | |
| n3 Fatty acids | | | | |
| 18:3n−3 | 1.96 ± 0.64 | 1.85 ± 0.95 | 1.94 ± 0.55 | 2.21 ± 1.18 |
| (mg/L) | | | | |
| 20:5n−3 | 2.28 ± 0.51 | 1.46 ± 0.36² | 2.88 ± 0.64 | 1.80 ± 0.61² |
| (mg/L) | | | | |
| 22:5n−3 | 15.8 ± 2.4 | 9.26 ± 1.55² | 17.7 ± 2.27 | 8.00 ± 2.02² |
| (mg/L) | | | | |
| 22:6n−3 | 37.1 ± 7.4 | 75.2 ± 11.1² | 23.7 ± 3.9 | 73.5 ± 11.9² |
| (mg/L) | | | | |
| Totals | | | | |
| Total SFA | 456 ± 66 | 447 ± 42 | 431 ± 43 | 439 ± 53 |
| (mg/L)⁴ | | | | |
| Total MUFA | 219 ± 26 | 204 ± 24² | 223 ± 19 | 221 ± 35 |
| (mg/L)⁵ | | | | |
| Total n−6 LCP | 243 ± 33 | 246 ± 24 | 241 ± 31 | 237 ± 32 |
| (mg/L) | | | | |
| Total n−3 LCP | 55.4 ± 9.1 | 86.2 ± 11.5² | 44.5 ± 5.3 | 83.5 ± 12.9² |
| (mg/L) | | | | |
| Ratios | | | | |
| DHA:n−6 DPA | 2.94 ± 0.59 | 9.14 ± 1.99² | 2.04 ± 0.30 | 12.7 ± 3.7² |
| n−6:n−3 LCP | 4.43 ± 0.59 | 2.87 ± 0.25² | 5.44 ± 0.54 | 2.87 ± 0.31² |
| Mead | 0.007 ± 0.003 | 0.004 ± 0.004² | 0.006 ± 0.003 | 0.004 ± 0.003² |
| acid:ARA | | | | |
| Unsaturation | 1820 ± 215 | 1950 ± 184³ | 1750 ± 185 | 1970 ± 251³ |
| index | | | | |

Notes:
¹mean ± SD. LCP, long-chain polyunsaturated fatty acids; AA, arachidonic acid; DPA, docosapentaenoic acid; DHA, docosahexaenoic acid; Mead acid, 20:3n9. The unsaturation index is the sum of the number of double bonds in each fatty acid the mass of each fatty acid for all the fatty acids.
^{2,3}Significantly different from the control-formula group: ²P < 0.001, ³P < 0.01.
⁴Includes 14:0, 16:0, 17:0, 18:0, 20:0, 22:0, and 24:0.
⁵Includes 14:1, 16:1, 18:1, 20:1, 22:1, and 24:1.

Both plasma and RBC concentrations of ARA were significantly higher at 17 weeks in infants who consumed LCP-supplemented formula than in those who consumed control formula. At 52 weeks, plasma concentrations of ARA were significantly higher in the infants who consumed LCP-supplemented formula than in those who consumed control formula, but RBC concentrations of ARA were not significantly different in the 2 diet groups. At 17 weeks, plasma and RBC concentrations of LA were significantly lower in the LCP-supplemented-formula group than in the control-formula group. At 52 weeks, there were no significant differences between the 2 diet groups in their LA concentrations in plasma or RBCs. In both plasma and RBCs, 20:3n6 and n6 DPA were lower in the LCP-supplemented-formula group than in the control-formula group at both 17 and 52 weeks; 22:4n6 concentrations in

RBCs but not in plasma were significantly lower in the LCP-supplemented-formula group than in the control-formula group at both ages.

The ratio of DHA to n-6 DPA was significantly lower whereas the ratio of n-6 to n-3 LCPs was significantly higher in the control-formula group than in the LCP-supplemented-formula group at both 17 and 52 weeks. The ratio of Mead acid (20:3n-9) to ARA was significantly higher in the control-formula group than in the LCP-supplemented-formula group at both 17 and 52 weeks, and the unsaturation index was significantly higher in the LCP-supplemented-formula group at both ages.

Growth:

Box plots of z scores for weight, length, and head circumference for both diet groups are shown in FIG. 6. All anthropometric out-come data were normally distributed. With the use of repeated-measures analysis of variance, no significant main effect of diet was found for weight, length, or head circumference. In addition, there were no significant differences between the diet groups in weight-for-length, subscapular fat, or triceps fat deposition (data not shown).

Sweep VEP Acuity:

Mean sweep VEP acuity for both randomized diet groups at each age is summarized in FIG. 7. All acuity outcome data were normally distributed. There were significant main effects of diet and of age and a significant interaction between them. In the planned comparisons, there were no significant differences between the 2 diet groups at 6 weeks of age, but acuity in the control-formula group was significantly poorer than in the LCP-supplemented group at 17, 26, and 52 weeks of age.

Random Dot Stereoacuity:

Mean random dot stereoacuity for both randomized diet groups at each age is summarized in FIG. 8. There was no significant main effect of diet. There was a significant main effect of age and a significant interaction between diet and age. In planned comparisons, the LCP-supplemented-formula group had significantly better stereoacuity than did the control-formula group at 17 weeks of age. There were no significant differences in stereoacuity between the control-formula group and the LCP-supplemented-formula group at 39 or 52 weeks of age.

Linear Regression of Visual Function Outcomes on the LCP Composition of Plasma and RBCs:

TABLE 11
Linear regression of visual function outcomes on the composition
of long-chain polyunsaturated fatty
acids in plasma and red blood cells (RBCs)¹

| VEP acuity | | | VEP acuity | | | Stereoacuity | | |
|---|---|---|---|---|---|---|---|---|
| at 17 weeks | | | at 52 weeks | | | at 17 weeks | | |
| n | r | P | n | r | P | n | r | P |

Plasma

| | | | | | | | | | |
|---|---|---|---|---|---|---|---|---|---|
| 18:2n−6 | 60 | 0.001 | NS | 58 | −0.28 | NS | 56 | 0.30 | NS |
| 20:4n−6 | 60 | −0.39 | <0.002 | 58 | −0.51 | <0.001 | 56 | −0.30 | NS |
| 18:3n−3 | 60 | 0.02 | NS | 58 | −0.28 | NS | 56 | 0.30 | NS |
| 22:6n−3 | 60 | −0.42 | <0.001 | 58 | −0.60 | <0.001 | 56 | −0.33 | <0.01 |

RBCs

| | | | | | | | | | |
|---|---|---|---|---|---|---|---|---|---|
| 18:2−n6 | 60 | 0.17 | NS | 58 | 0.05 | NS | 56 | 0.43 | <0.001 |
| 20:4−n6 | 60 | −0.25 | NS | 58 | −0.33 | <0.001 | 56 | −0.22 | NS |
| 18:3−n3 | 60 | 0.07 | NS | 58 | 0.09 | NS | 56 | 0.27 | NS |
| 22:6−n3 | 60 | −0.32 | <0.01 | 58 | −0.61 | <0.001 | 56 | −0.31 | NS |

Notes:
¹VEP, visual evoked potential.

The relation between the LCP composition of plasma and RBCs and sweep VEP acuity at 17 and 52 weeks was examined by linear regression (Table 11). Because sweep VEP acuities were expressed in log MAR, negative regression coefficients would indicate that better acuity is associated with a higher concentration of LCPs whereas positive regression coefficients would indicate that poorer acuity is associated with a higher concentration of LCPs. Better sweep VEP acuity at 17 and 52 weeks was associated with higher plasma concentrations of DHA and ARA. Neither LA nor α-LNA concentrations in plasma were associated significantly with sweep VEP acuity at either age. In RBCs, better sweep VEP acuity at 17 weeks was only weakly associated with DHA concentration. At 52 weeks, sweep VEP acuity was associated with higher concentrations of both DHA and ARA in RBCs. Neither LA nor ALNA concentrations in RBCs were significantly associated with sweep VEP acuity at either age. The relation between the LCP composition of plasma and RBCs and stereoacuity at 17 weeks was also examined by linear regression (Table 11). Because stereoacuity was expressed in logs, negative regression coefficients would indicate that better stereoacuity is associated with a higher concentration of LCPs whereas positive regression coefficients would indicate that poorer stereoacuity is associated with a higher concentration of LCPs. Better stereoacuity at 17 weeks was associated with higher plasma DHA concentrations. ARA, LA, and α-LNA concentrations in plasma were not significantly associated with stereoacuity. In RBCs, a higher concentration of LA at 17 weeks was associated with poorer stereoacuity. DHA, ARA, and α-LNA concentrations in RBCs were not significantly associated with stereoacuity.

Brief Discussion of Results

The results from the present study suggest that the critical period during which the dietary supply of LCPs may influence the maturation of cortical function in term infants extends beyond the first 6 weeks of life. Despite a dietary supply of LCPs from breast-feeding during the first 6 weeks, infants who were randomly assigned to receive control formula after weaning showed poorer functioning of the visual cortex than did infants who were randomly assigned to receive formula supplemented with 0.36% DHA and 0.72% ARA.

Both formulas were well tolerated by infants; the only intolerance, which was noted in 4 infants, was related to symptoms suggestive of intolerance to lactose or cow milk protein and occurred in both diet groups. Moreover, there were no significant differences in growth between the 2 diet groups. There was a trend for both diet groups to be slightly larger (both in weight and length) than the CDC's normative cohort; this probably reflects the eligibility criterion of birth weight, 2800 g, a working definition of the appropriate weight for a full-term birth, compared with the CDC eligibility criterion of 1500 g.

Consumption of LCP-supplemented formula by term infants resulted in higher plasma and RBC concentrations of DHA than did consumption of control formula; these higher concentrations are more like those of breast-fed term infants. The lower plasma and RBC concentrations of LA in the LCP-supplemented-formula group compared with the control-formula group at 17 weeks may reflect, in part, displacement of LA by both DHA and ARA. By 52 weeks of age, the lower concentration of LA was no longer evident, possibly because of the introduction of solid foods and the concomitant reduction in study formula intake.

Plasma ARA concentrations were higher in the LCP-supplemented-formula group throughout the study period, and RBC concentrations of ARA were higher at 17 but not at 52 weeks of age. This suggests that the infants who received control formula may have synthesized sufficient ARA sometime after 17 weeks of age. Low concentrations of ARA in plasma and RBCs are associated with poorer growth in preterm infants; thus, it may be prudent to provide dietary supplementation of ARA in conjunction with DHA to maintain a balanced ratio of n3 to n6 LCPs similar to that present in human milk.

A small but significant reduction in the unsaturation index was found in the control-formula group throughout the study period. Changes in the unsaturation index can influence the function of various membrane-related enzymes, receptors, and nutrient transport systems. A higher ratio of Mead acid to ARA was also present in the control-formula group. This finding is consistent with an excess conversion of oleic acid (18:1n9) to Mead acid and is suggestive of essential fatty acid insufficiency.

Although there was no significant difference in sweep VEP acuity between the 2 groups of infants at the last visit before weaning, a clear difference was present at 9-11 weeks after weaning (at the 17-weeks visit), and the acuity difference persisted at 26 and 52 weeks of age. The average difference between the LCP-supplemented formula and control-formula groups is equivalent to one line on an eye chart; e.g., at 52 weeks of age, the Snellen equivalents of the LCP-supplemented-formula and control-formula groups are 20/30 and 20/40, respectively.

In an earlier study of term infants fed the same control or LCP-supplemented formulas from birth through 17 weeks of age, infants who consumed LCP-supplemented formula had better VEP acuity at 17 and 52 weeks of age but not at 26 weeks of age. See, Birch et al. (1998), opus cit. A comparison of acuity results from both studies is provided in FIG. 9. In the present study, the acuity of both groups of infants at 6 weeks (when they were breast-feeding) was better than the acuity of the control-formula group in the earlier study and similar to the acuity of the infants fed formula supplemented with DHA and ARA. At both 17 and 52 weeks, there is good agreement between the 2 studies. It is only at 26 weeks that there is a significant difference in the outcomes of the 2 studies. In the present study, the LCP-supplemented formula group had somewhat better acuity than in the earlier study, whereas the control formula group had somewhat poorer acuity than in the earlier study. It is possible that continued feeding of LCP-supplemented formula beyond 4 months of age enhanced the development of the visual cortex. It is also possible that the initial 6 weeks of LCP supply via breast-feeding before the initiation of formula feeding had an imprinting effect that altered the effects of subsequent LCP-supplemented or control formula on the maturation of the visual cortex.

Random dot stereoacuity has not been used previously as an outcome measure for the visual cortex in randomized clinical trials of infant LCP nutrition. Random dot stereopsis reflects processing in the visual cortex because it relies on a combination of monocular inputs that lack any monocular form information. Random dot stereoacuity is not present before 3 months of age in healthy infants but matures much more rapidly than does acuity at 3-5 months of age; it should be especially sensitive to differences in the maturation of the visual cortex during this period of infancy. This prediction was supported in the present study by the finding of better random dot stereoacuity at 17 weeks of age in infants who consumed LCP-supplemented formula than in infants who consumed control formula.

The embodiment of the present invention thus addresses the need for safe and effective alternatives to breast-feeding after weaning to infant formula. The results presented in this example shows that LCP-supplemented formula is well tolerated and beneficial to the maturation of the visual cortex in term infants weaned at 6 weeks of age.

### EXAMPLE 3

This example shows a particular infant formula that may be used in the present invention. The nutrient composition of this particular formula is shown in Table 12. The formula comprises the following ingredients: reduced minerals whey, nonfat milk, vegetable oil (palm olein, soy, coconut, and high oleic sunflower oils), lactose, and less than 1% of each of the following components: mortierella alpina oil (a source of ARA), crypthecodinium cohnii oil (a source of DHA), mono- and diglycerides, soy lecithin, carrageenan, vitamin A palmitate, vitamin D₃, vitamin E acetate, vitamin K₁, thiamin hydrochloride, vitamin B₆ hydrochloride, vitamin B₁₂, niacinamide, folic acid, calcium pantothenate, biotin, sodium ascorbate, ascorbic acid, inositol, calcium chloride, calcium phosphate, ferrous sulfate, zinc sulfate, manganese sulfate, cupric sulfate, sodium chloride, sodium citrate, potassium citrate, potassium hydroxide, sodium selenite, taurine, and nucleotides (adenosine 5′-monophosphate, cytidine 5′-monophosphate, disodium guanosine 5′-monophosphate, disodium uridine 5′-monophosphate).

The formula content of DHA is about 17 mg/100 kcal and the formula content of ARA is about 34 mg/100 kcal.

TABLE 12
Term Infant Formula Nutrient Levels (per 100 Cal)

| Nutrient | Unit | Term Formula |
|---|---|---|
| Protein | g | 2.1 |
| Fat | g | 5.3 |
| Linoleic Acid | mg | 860 |
| Carbohydrate | g | 10.9 |
| Vitamin A | IU | 300 |
| Vitamin D | IU | 60 |
| Vitamin E | IU | 2 |
| Vitamin K | μg | 8 |
| Thiamin (vitamin B1) | μg | 80 |
| Riboflavin (vitamin B2) | μg | 140 |
| Vitamin B6 | μg | 60 |
| Vitamin B12 | μg | 0.3 |
| Niacin | μg | 1000 |
| Folic acid (folacin) | μg | 16 |
| Pantothenic acid | μg | 500 |
| Biotin | μg | 3 |
| Vitamin C (ascorbic acid) | mg | 12 |
| Choline | mg | 12 |
| Inositol | mg | 6 |
| Calcium | mg | 78 |
| Phosphorus | mg | 53 |
| Magnesium | mg | 8 |
| Iron | mg | 1.8 |
| Zinc | mg | 1 |
| Manganese | μg | 15 |
| Copper | μg | 75 |
| Iodine | μg | 10 |
| Selenium | μg | 2.8 |
| Sodium | mg | 27 |
| Potassium | mg | 108 |
| Chloride | mg | 63 |

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by the authors and no admission is made that any reference constitutes prior art. Applicants reserve the right to challenge the accuracy and pertinency of the cited references.

Although various embodiments of the invention have been described using specific terms, devices, and methods, such description is for illustrative purposes only. The words used are words of description rather than of limitation. It is to be understood that changes and variations may be made by those of ordinary skill in the art without departing from the spirit or the scope of the present invention, which is set forth in the following claims. In addition, it should be understood that aspects of the various embodiments may be interchanged both in whole or in part. Therefore, the spirit and scope of the appended claims should not be limited to the description of the preferred versions contained therein.

What is claimed is:

1. A method for enhancing the visual development of term infants that have been breast-fed to an age of from about one and one-half months to about six and one-half months of age before weaning to formula, the method comprising administering to those infants a visual development enhancing amount of DHA and ARA from the time of weaning.

2. The method of claim 1 wherein DHA and ARA are supplemented into infant formulas.

3. The method of claim 1 wherein the ratio of ARA:DHA is from about 1:3 to about 4:1.

4. The method of claim 1 wherein the ratio of ARA:DHA is from about 1:2 to about 3:1.

5. The method of claim 1 wherein the ratio of ARA:DHA is from about 1:1 to about 2:1.

6. The method of claim 1 wherein the ratio of ARA:DHA is about 2:1.

7. The method of claim 1 wherein the amount of DHA administered to the infant is from about 11 mg to about 75 mg per kg of the infant's body weight per day.

8. The method of claim 1 wherein the amount of DHA administered to the infant is from about 12 mg to about 60 mg per kg of the infant's body weight per day.

9. The method of claim 1 wherein the amount of DHA administered to the infant is from about 13 mg to about 50 mg per kg of the infant's body weight per day.

10. The method of claim 1 wherein the amount of DHA administered to the infant is from about 15 mg to about 40 mg per kg of the infant's body weight per day.

11. The method of claim 1 wherein the amount of DHA administered to the infant is from about 15 mg to about 26 mg per kg of the infant's body weight per day.

12. The method of claim 2 wherein the infant formula comprises DHA in an amount of about 12 mg/100 kcal to about 50 mg/100 kcal and ARA in an amount of about 12 mg/100 kcal to about 100 mg/100 kcal.

13. The method of claim 2 wherein the infant formula comprises DHA in an amount of about 13 mg/100 kcal to about 33 mg/100 kcal and ARA in an amount of about 13 mg/100 kcal to about 67 mg/100 kcal.

14. The method of claim 2 wherein the infant formula comprises DHA in an amount of about 15 mg/100 kcal to about 20 mg/100 kcal and ARA in an amount of about 15 mg/100 kcal to about 40 mg/100 kcal.

15. The method of claim 1 wherein the infants are administered a visual development enhancing amount of DHA and ARA for at least 1 month after weaning to formula.

16. The method of claim 1 wherein the infants are administered a visual development enhancing amount of DHA and ARA for at least 2 months after weaning to formula.

17. The method of claim 1 wherein the infants are administered a visual development enhancing amount of DHA and ARA for at least 6 months after weaning to formula.

18. The method of claim 1 wherein the infants are administered a visual development enhancing amount of DHA and ARA for at least 9 months after weaning to formula.

19. The method of claim 1 wherein the infants are administered a visual development enhancing amount of DHA and ARA for at least 12 months after weaning to formula.

20. A method for enhancing the visual development of term infants that have been breast-fed to an age of from about one and one-half months to about six and one-half months, the method comprising administering to those infants a visual development enhancing amount of DHA and ARA from the time of weaning until the infants are at least one year of age.

21. The method of claim 20 wherein the ratio of ARA:DHA is from about 1:3 to about 4:1.

22. The method of claim 20 wherein the ratio of ARA:DHA is from about 1:2 to about 3:1.

23. The method of claim 20 wherein the ratio of ARA:DHA is from about 1:1 to about 2:1.

24. The method of claim 20 wherein the ratio of ARA:DHA is about 2:1.

25. The method of claim 20 wherein the amount of DHA administered to the infant is from about 11 mg to about 75 mg per kg of the infant's body weight per day.

26. The method of claim 20 wherein the amount of DHA administered to the infant is from about 12 mg to about 60 mg per kg of the infant's body weight per day.

27. The method of claim 20 wherein the amount of DHA administered to the infant is from about 13 mg to about 50 mg per kg of the infant's body weight per day.

28. The method of claim 20 wherein the amount of DHA administered to the infant is from about 15 mg to about 40 mg per kg of the infant's body weight per day.

29. The method of claim 20 wherein the amount of DHA administered to the infant is from about 15 mg to about 26 mg per kg of the infant's body weight per day.

30. The method of claim 20 wherein DHA and ARA are supplemented into an infant formula.

31. The method of claim 30 wherein the infant formula comprises DHA in an amount of about 12 mg/100 kcal to about 50 mg/100 kcal and ARA in an amount of about 12 mg/100 kcal to about 100 mg/100 kcal.

32. The method of claim 30 wherein the infant formula comprises DHA in an amount of about 13 mg/100 kcal to about 33 mg/100 kcal and ARA in an amount of about 13 mg/100 kcal to about 67 mg/100 kcal.

33. The method of claim 30 wherein the infant formula comprises DHA in an amount of about 15 mg/100 kcal to about 20 mg/100 kcal and ARA in an amount of about 15 mg/100 kcal to about 40 mg/100 kcal.

34. A method for enhancing the visual development of term infants that have been breast-fed to an age of from about one and one-half months, to about six and one-half months, the method comprising feeding the infants after weaning with an infant formula comprising fats, proteins, carbohydrates, and a visual development enhancing amount of ARA and DHA.

35. The method of claim 34 wherein the infant formula comprises DHA in an amount of about 12 mg/100 kcal to about 50 mg/100 kcal and ARA in an amount of about 12 mg/100 kcal to about 100 mg/100 kcal.

36. The method of claim 34 wherein the infant formula comprises DHA in an amount of about 13 mg/100 kcal to about 33 mg/100 kcal and ARA in an amount of about 13 mg/100 kcal to about 67 mg/100 kcal.

37. The method of claim 34 wherein the infant formula comprises DHA in an amount of about 15 mg/100 kcal to about 20 mg/100 kcal and ARA in an amount of about 15 mg/100 kcal to about 40 mg/100 kcal.

38. The method of claim 34 wherein the infants are fed the DHA- and ARA-supplemented formula until at least the age of 6.5 months.

39. The method of claim 34 wherein the infants are fed the DHA- and ARA-supplemented formula until at least the age of 12 months.
